(19) United States
(12) Patent Application Publication
(10) Pub. No.: US 2026/0232836 A1
(43) Pub. Date: Aug. 13, 2026

(54) Peptide-Conjugated Lipid Nanoparticle (LNP) Compositions and Methods for Brain-Targeted Delivery of Therapeutic Agents

(71) Applicant: THE TRUSTEES OF THE UNIVERSITY OF PENNSYLVANIA, Philadelphia, PA (US)

(72) Inventors: Michael J. Mitchell, Philadelphia, PA (US); Emily Han, Philadelphia, PA (US)

(21) Appl. No.: 19/366,029

(22) Filed: Oct. 22, 2025

Related U.S. Application Data
(60) Provisional application No. 63/710,179, filed on Oct. 22, 2024.

(51) Int. Cl. A61K 47/69; A61K 31/7105; A61K 47/64; C12N 9/22; C12N 15/11; C12N 15/88

(52) U.S. Cl. CPC A61K 47/6911; A61K 31/7105; A61K 47/64; C12N 9/222; C12N 15/11; C12N 15/88; C12N 2310/20

(57) ABSTRACT

The present disclosure relates, in part, to polypeptide-conjugated lipid nanoparticle (LNP) compositions, and methods of use thereof for brain-targeted delivery of therapeutic cargo. In another aspect, the disclosure provides methods for treating, preventing, and/or ameliorating disease and/or disorders of the brain (e.g., neurological disease and/or disorders).

### CROSS-REFERENCE TO RELATED APPLICATIONS

This application claims priority under 35 U.S.C. § 119(e) to U.S. Provisional Patent Application No. 63/710,179, filed Oct. 22, 2024, which is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### REFERENCE TO AN ELECTRONIC SEQUENCE LISTING

The XML file named “046483-7384US1—Sequence Listing.xml” created
Oct. 22, 2025, comprising 4,693 bytes, is incorporated herein by reference in its entirety.

### BACKGROUND

Delivering nucleic acid therapeutics to the brain remains a significant challenge, in part due to the blood-brain barrier (BBB). The BBB prevents about 98% of small molecule drugs and about 100% of large molecule drugs, including naked messenger RNA (mRNA), from entering the brain. Ionizable lipid nanoparticles (LNPs) have emerged as the most clinically advanced nucleic acid delivery vehicle following the FDA approval of Onpattro and the COVID-19 vaccines. However, for applications beyond liver-based therapies and vaccines, achieving tissue-specific delivery has proven difficult with LNPs, with research efforts being directed to active targeting to overcome this challenge. Examples of active targeting strategies for tissue- or cell-specific LNP delivery include functionalization with antibodies targeting the lungs, T cells, hematopoietic stem cells, bone marrow in multiple myeloma, cancer cells and tumor myeloid cells, the placenta, and inflamed cerebral vasculature. While antibodies have high binding affinity, they also have limitations including immunogenicity, susceptibility to proteases, large size, difficulty achieving a binding-specific orientation, and high production costs.

Thus, there is a need in the art for active targeting strategies which facilitate transport of LNPs and/or therapeutic cargo thereof across the blood-brain barrier. The present disclosure addresses this unmet need.

### BRIEF SUMMARY

In one aspect, the disclosure provides a lipid nanoparticle (LNP) composition comprising:
(a) at least one ionizable lipid;
(b) at least one neutral lipid;
(c) cholesterol lipid and/or a modified derivative thereof,
(d) at least one polymer conjugated lipid and/or a modified derivative thereof, and
(e) a cell targeting polypeptide which specifically binds to a receptor overexpressed on the surface of a brain endothelial cell or neuron, optionally wherein the cell targeting domain is covalently conjugated to at least one component of the LNP.

In another aspect, the disclosure provides a pharmaceutical composition comprising the lipid nanoparticle (LNP) of the disclosure and at least one pharmaceutically acceptable carrier.

In another aspect, the disclosure provides a method of treating, preventing, and/or ameliorating a disease or disorder in a subject. In certain embodiments, the method comprises administering to the subject the LNP of the disclosure or a pharmaceutical composition thereof.

In another aspect, the disclosure provides a method of delivering at least one cargo molecule to a brain endothelial cell or neuronal cell of a subject. In certain embodiments, the method comprises administering to the subject the LNP of the disclosure or a pharmaceutical composition thereof.

### BRIEF DESCRIPTION OF THE FIGURES

The drawings illustrate generally, by way of example, but not by way of limitation, various embodiments of the present application.

FIGS. 1A-1E: Formulation and characterization of peptide-functionalized LNPs for targeted mRNA delivery to the brain. FIG. 1A: Schematic overview depicting the process of engineering and validating targeted LNPs for brain delivery following systemic administration. FIG. 1B: Schematic showing the formulation process for targeted LNPs. FIG. 1C: Size of LNPs on days 0 and 5 post-formulation. Data is shown as mean+SEM, n=2 formulation replicates each with n=3 technical replicates. FIG. 1D: Zeta potential of LNPs. Data is shown as mean+SEM, n=2 formulation replicates each with n=3 technical replicates. FIG. 1E Encapsulation efficiency of LNPs. Data is shown as mean+SEM, n=2 formulation replicates.

FIGS. 2A-2B: In vitro transfection efficacy of peptide-functionalized LNPs in brain endothelial and neuronal cells. FIG. 2A: Luciferase expression in LNP-treated brain endothelial and neuronal cells cultured in serum-free media. Cells were treated with LNPs for 24 h at 60 ng mRNA/20 k cells. Data is shown as mean+SEM, n=2 biological replicates each with n=4 technical replicates. FIG. 2B: Luciferase expression in LNP-treated brain endothelial and neuronal cells cultured in 10% FBS-supplemented media. Cells were treated with LNPs for 24 h at 60 ng mRNA/20 k cells. Data is shown as mean+SEM, n=3 biological replicates each with n=4 technical replicates. All data is normalized to the untargeted 0% lipid-PEG-mal substitution group. One-way ANOVA with Dunnett's multiple comparisons test was used to determine statistical significance compared to the 0% lipid-PEG-mal substitution group, *p<0.05, **p<0.01, ***p<0.001, ****p<0.0001.

FIGS. 3A-3F: Peptide-functionalized LNP transfection in a BBB transwell and exosome secretion model. FIG. 3A: Schematic depicting the experimental approach for establishing the transwell model. FIG. 3B: Luciferase expression in apical brain endothelial cells treated with LNPs for 24 h at 60 ng mRNA/20 k endothelial cells. FIG. 3C: Luciferase expression in basolateral neuronal cells after 24 h. Data is shown as mean+SEM, n=3 technical replicates.

FIG. 3D: Schematic depicting the experimental procedure for evaluating the potential of endothelial cell-secreted exosomes to transfect neuronal cells. FIG. 3E: Luciferase expression in brain endothelial cells treated with LNPs for 2 h at 60 ng mRNA/20 k cells. FIG. 3F: Luciferase expression in neuronal cells treated with endothelial cell-derived exosomes for 24 h. Data is shown as mean+SEM, n=4 technical replicates. All data is normalized to the untargeted 0% lipid-PEG-mal substitution group. One-way ANOVA with Dunnett's multiple comparisons test was used to determine statistical significance compared to the 0% lipid-PEG-mal substitution group, *p<0.05, **p<0.01, ***p<0.001, ****p<0.0001.

FIGS. 4A-4E: In vivo mRNA transfection after intravenous administration of peptide-functionalized LNPs. FIGS. 4A-4B: Representative in vivo imaging system (IVIS) images (FIG. 4A) and quantification (FIG. 4B) of luciferase mRNA LNP delivery to the brain in adult C57BL/6 mice. Untargeted LNPs have 0% lipid-PEG-mal substitution, targeted LNPs have 10% lipid-PEG-mal substitution. Mice were injected intravenously with LNPs at a dose of 0.3 mg/kg mRNA or PBS and sacrificed after 6 h. Representative IVIS images are shown from the mice with the greatest luciferase expression in the brain for each group. Relative radiance was calculated by subtracting PBS group luminescence from LNP groups. FIGS. 4C-4D: Brain to liver (FIG. 4C) and spleen to liver (FIG. 4D) luminescence ratios for LNP-treated mice. Data is shown as mean+SD with n=4 mice. One-way ANOVA with Dunnett's multiple comparisons test was used to determine statistical significance compared to the 0% substitution group, ***p<0.001, ****p<0.0001. FIG. 4E: Distribution of luminescence across brain, heart, lungs, liver, kidneys, and spleen. Data is shown as % of total luminescence=(organ luminescence)/(total organ luminescence)*100.

FIGS. 5A-5H: Cell-specific flow cytometric analysis of in vivo LNP delivery to the brain. FIGS. 5A-5H: The percentage of mCherry⁺ cells in live cells (FIG. 5A), neurons (FIG. 5B), astrocytes (FIG. 5C), and endothelial cells (FIG. 5D) and the percentage of DiR⁺ cells in live cells (FIG. 5E), neurons (FIG. 5F), astrocytes (FIG. 5G), and endothelial cells (FIG. 5H) from adult C57BL/6 mice treated with mCherry mRNA LNPs containing 1 mol % DiR. Untargeted LNPs have 0% lipid-PEG-mal substitution, peptide-functionalized LNPs have 10% lipid-PEG-mal substitution. Mice were injected intravenously with LNPs at a dose of 0.3 mg/kg mRNA or PBS and sacrificed after 12 h. Bar graphs are reported as mean+SD with n=4 mice. One-way ANOVA with Tukey's multiple comparisons test was used to determine statistical significance, *p<0.05, **p<0.01, ***p<0.001, ****p<0.0001.

FIG. 6: Polydispersity index (PDI) of peptide-functionalized LNPs. Data is shown as mean+SEM, n=2 formulation replicates each with n=3 technical replicates. Missing bar indicates a measurement was not able to be recorded by the plate reader.

FIGS. 7A-7B: Peptide concentration of peptide-functionalized LNPs pre- and post-purification of excess peptides. Peptide concentration was calculated using a FluoProdige Protein Quantification Assay Kit (FIG. 7A) and a CBQCA Protein Quantitation Kit (FIG. 7B). All LNPs have 10% lipid-PEG-mal substitution. Peptide concentration of peptide-functionalized LNPs was calculated by subtracting the fluorescence of the no peptide LNP and then converting the fluorescence to concentration using a BSA standard curve. Data is shown as mean+SEM, n=3 technical replicates.

FIG. 8: Cell viability of endothelial and neuronal cells treated with peptide-functionalized LNPs. Cells were treated with LNPs for 24 h at 60 ng mRNA/20 k cells. Data is shown as mean+SEM, n=4 technical replicates, normalized to an untreated group.

FIG. 9: Transfection of endothelial and neuronal cells by peptide-functionalized LNPs after human serum incubation. LNPs were incubated in 40% (v/v) human serum for 30 minutes at 37° C. Afterwards, cells were treated with LNPs for 24 h at 60 ng mRNA/20 k cells. Data is shown as mean+SEM, n=2 biological replicates each with n=4 technical replicates. mApoE LNPs with 30% and 50% lipid-PEG-mal substitution were not tested due to low mRNA concentration. Data is normalized to the untargeted 0% lipid-PEG-mal substitution group with human serum incubation.

FIG. 10: Dose-dependent transfection of endothelial and neuronal cells treated with peptide-functionalized LNPs. Untargeted LNPs have 0% lipid-PEG-mal substitution, targeted LNPs have 10% lipid-PEG-mal substitution. Cells were treated with LNPs for 24 h at 20, 40, or 60 ng mRNA/20 k cells. Data is shown as mean+SEM, n=4 technical replicates.

FIG. 11: LNP-mediated mCherry expression in hCMEC/D3 endothelial cells. Untargeted LNPs have 0% lipid-PEG-mal substitution, RVG29 LNPs have 10% lipid-PEG-mal substitution. Cells were treated with LNPs for 24 h at 30 ng mRNA/20 k cells. Scale bar is 100 m.

FIG. 12: LNP-mediated mCherry expression in SH-SY5Y neuronal cells. Untargeted LNPs have 0% lipid-PEG-mal substitution, mApoE LNPs have 10% lipid-PEG-mal substitution. Cells were treated with LNPs for 24 h at 30 ng mRNA/20 k cells. Scale bar is 100 m.

FIG. 13: Intracellular distribution of peptide-functionalized LNPs over time in hCMEC/D3 endothelial cells. Untargeted LNPs have 0% lipid-PEG-mal substitution, peptide-functionalized LNPs have 10% lipid-PEG-mal substitution. Cells were treated with LNPs containing luciferase mRNA and 5 mol % DiR for 2 h at 60 ng mRNA/20 k cells, and then, following a media change, were incubated for an additional 0, 2, 10, or 22 h, leading to total incubation times of 2, 4, 12, or 24 h. Prior to imaging, cells were stained with Hoechst and LysoTracker to visualize nuclei and acidic compartments like late endosomes and lysosomes, respectively. Scale bar is 25 m.

FIGS. 14A-14B: IVIS luminescence imaging (FIG. 14A) and quantification (FIG. 14B) of organs from in vivo study with peptide-functionalized LNPs containing luciferase mRNA. Untargeted LNPs have 0% lipid-PEG-mal substitution, targeted LNPs have 10% lipid-PEG-mal substitution. Mice were injected intravenously with LNPs at a dose of 0.3 mg/kg mRNA or PBS and sacrificed after 6 h. Data is reported as mean+SD with n=4 mice. Data is from the same experiment as in FIGS. 4A-4E.

FIG. 15: Representative gating strategy for flow cytometric analysis of mouse brain cells. From the live cell population, neurons were defined as NeuN⁺, endothelial cells were defined as CD31+/GFAP−/NeuN−, and astrocytes were defined as GFAP+/CD31−/NeuN−.

FIG. 16:. mCherry and DiR mean fluorescence intensity (MFI) of live cells, neurons, endothelial cells, and astrocytes from flow cytometric analysis of brain cells. Untargeted LNPs have 0% lipid-PEG-mal substitution, peptide-functionalized LNPs have 10% lipid-PEG-mal substitution. Mice were injected intravenously with LNPs containing mCherry mRNA and 1 mol % DiR at a dose of 0.3 mg/kg mRNA or PBS and sacrificed after 12 h. Data is reported as mean+SD with n=4 mice. All data was normalized by subtracting the background fluorescence from PBS-treated groups. One-way ANOVA with Tukey's multiple comparisons test was used to determine statistical significance, *p<0.05, **p<0.01, ***p<0.001, ****p<0.0001. Data is from the same experiment as FIGS. 5A-5H.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

As indicated elsewhere herein, tissue specific delivery of therapeutic cargo, beyond liver-based therapies and/or vaccines, has proven difficult utilizing LNPs, and research efforts have directed to active targeting strategies to overcome this challenge (e.g., antibody-functionalized LNPs). While antibodies have high binding affinity, they also have limitations including immunogenicity, susceptibility to proteases, large size, difficulty achieving a binding-specific orientation, and high production costs.

Peptides are alternative targeting ligands that address many of these issues. They are comprised of amino acids, enabling higher functionalization density on the nanoparticle surface for improved binding affinity, and compared to antibodies, have lower immunogenicity and production costs. Recent work has demonstrated the ability of peptides to facilitate targeted mRNA LNP delivery to retinal neurons in mice and non-human primates. The potential of peptides to target LNPs to other tissues remains unexplored.

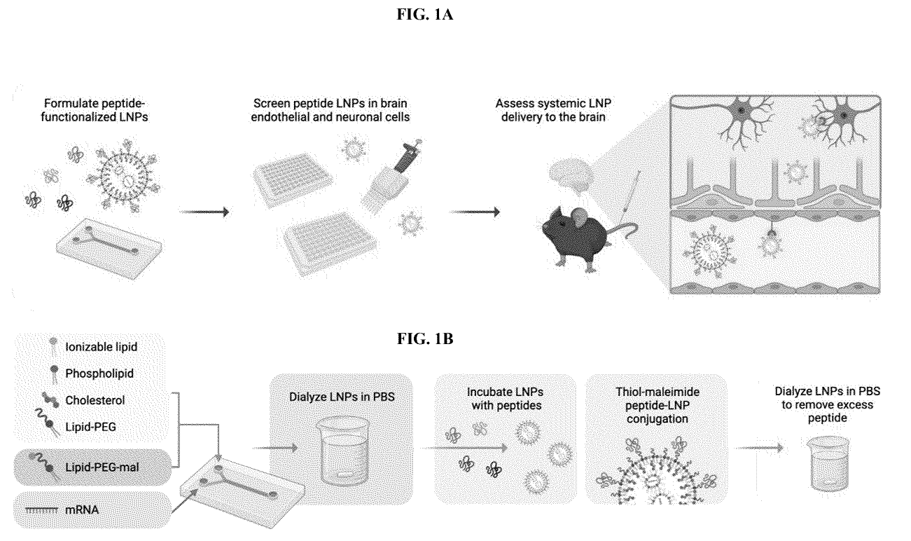

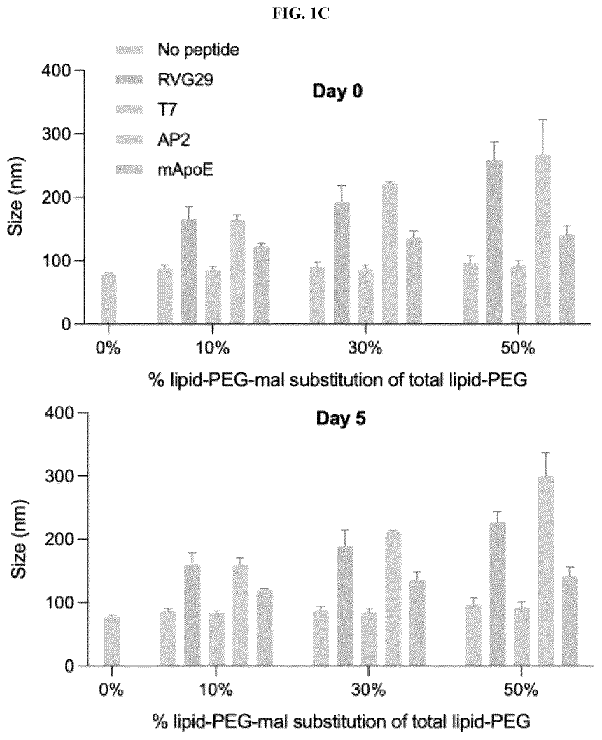

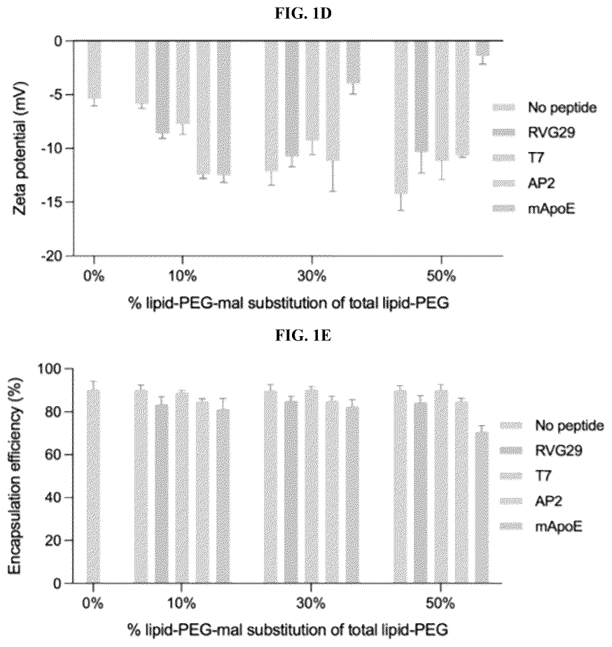

In one aspect, the disclosure describes the design of peptide-functionalized LNPs (pLNPs) for systemic mRNA delivery to the brain, targeting receptors highly expressed on both brain endothelial cells and neurons (FIG. 1A). The four non-limiting peptides have improved brain delivery of other nanoparticle systems: RVG29 targeting the nicotinic acetylcholine receptor, T7 targeting the transferrin receptor, angiopep-2 (AP2) targeting low density lipoprotein receptor-related protein 1 (LRP-1), and mApoE targeting low density lipoprotein receptor (LDLR).

mRNA LNPs were functionalized with these peptides at various surface densities via a thiol-maleimide click chemistry reaction. pLNPs demonstrated improved transfection efficiency in cultured brain endothelial and neuronal cells. In a co-culture transwell model, pLNPs were able to cross the endothelial monolayer and improve transfection of basolateral neuronal cells. It was demonstrated that exosomes secreted by pLNP-treated brain endothelial cells can transfect neuronal cells. Finally, it was demonstrated that pLNPs can transfect the mouse brain after systemic administration, and that specifically RVG29 LNPs significantly improve neuronal transfection compared to untargeted LNPs. Overall, these pLNPs may serve as a platform for delivering mRNA therapeutics across the BBB with potential applications in treating neurological disorders.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein, refer to a divalent radical of the moiety corresponding to the base group (e.g., heteroalkyl, cycloalkyl, and/or heterocycloalkyl). A divalent radical possesses two open valencies at any position(s) of the group, wherein each radical may be on a carbon atom or heteroatom. Thus, the divalent radical may form a single bond to two distinct atoms or groups, or may form a double bond with one atom.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “monovalent cation” as used herein refers to any positively charged (+1) organic or inorganic ion. Non-limiting examples include H⁺, NH₄⁺, Li⁺, Na⁺, K⁺, Cu⁺, Ag⁺, Cs⁺, and Au⁺.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cationic lipids comprise a protonatable tertiary amine (e.g., pH titratable) head group, C₁₈ alkyl chains, ether linkages between the head group and alkyl chains, and 0 to 3 double bonds. Such lipids include, e.g., DSDMA, DLinDMA, DLenDMA, and DODMA.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “monovalent” as used herein refers to a substituent connecting via a single bond to a substituted molecule. When a substituent is monovalent, such as, for example, F or Cl, it is bonded to the atom it is substituting by a single bond.

The term “mRNA” or “messenger RNA” as used herein refers to a ribonucleic acid sequences which encodes a peptide or protein. In certain embodiments, the mRNA may comprise a “transcript” that is produced by using a DNA template and encodes a peptide or protein. Typically, mRNA comprises 5′-UTR, protein coding region and 3′-UTR. mRNA can be produced by in vitro transcription from a DNA template. Methods of in vitro transcription are known to those of skill in the art. For example, various in vitro transfer kits are commercially available. According to the present invention, mRNA can be modified by further stabilizing modifications and cap formation in addition to the modifications according to the invention.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “siRNA” or “small interfering RNA” as used herein refers to a small (e.g. generally less than 30 nucleotides) non-coding RNA molecule which functions in transcriptional and post-transcriptional regulation of gene expression. Generally, a siRNA specifically targets 1 nucleic acid. In general, a siRNA comprises a double-stranded RNA molecule that ranges from about 15 to about 29 nucleotides in length. In some embodiments, the siRNA may be 16, 17, 18, 19, 20, 21, 22, 23, 24, 25, 26, 27, 28 or 29 nucleotides in length. In some embodiments, the siRNA may be less than 16, 17, 18, 19, 20, 21, 22, 23, 24, 25, 26, 27, 28 or 29 nucleotides in length. In some embodiments, the siRNA may be more than 16, 17, 18, 19, 20, 21, 22, 23, 24, 25, 26, 27, 28 or 29 nucleotides in length. A siRNA may optionally further comprise one or two single-stranded overhangs, e.g., a 5′ overhang on one or both ends, a 3′ overhang on one or both ends, or a combination thereof. The siRNA may be formed from two RNA molecules that hybridize together or, alternatively, may be generated from a short hairpin RNA (shRNA). In some embodiments, the two strands of the siRNA may be completely complementary, such that no mismatches or bulges exist in the duplex formed between the two sequences. In other embodiments, the two strands of the siRNA may be substantially complementary, such that one or more mismatches and/or bulges may exist in the duplex formed between the two sequences. In certain embodiments, one or both of the 5′ ends of the siRNA may have a phosphate group, while in other embodiments one or both of the 5′ ends lack a phosphate group. In other embodiments, one or both of the 3′ ends of the siRNA may have a hydroxyl group, while in other embodiments one or both of the 5′ ends lack a hydroxyl group. Typically, siRNAs are targeted to exonic sequences of the target nucleic acid. One strand of the siRNA, which is referred to as the “antisense strand” or “guide strand,” includes a portion that hybridizes with a target nucleic acid. A target nucleic acid refers to a nucleic acid sequence expressed by a cell for which it is desired expression be disrupted. In the context of a therapeutic composition of the invention, disrupting expression of a target nucleic acid may produce a beneficial effect. Those of skill in the art are familiar with programs, algorithms, and/or commercial services that design siRNAs for target genes. For example, the Rosetta siRNA Design Algorithm (Rosetta Inpharmatics, North Seattle, Wash.), MISSION® siRNA (Sigma-Aldrich, St. Louis, Mo.) and siGENOME siRNA (Thermo Scientific) may be used.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, O-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In one embodiment, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

### Lipids

In one aspect, the present disclosure provides an ionizable lipid of Formula (I), or a salt, solvate, stereoisomer, or isotopologue thereof:

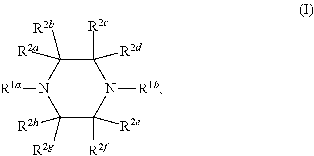

wherein:
R^{1a} and R^{1b} are each independently

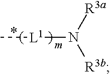

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L¹ is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L¹ is —CH₂—. In certain embodiments, L¹ is —(CH₂)₂—. In certain embodiments, L¹ is —(CH₂)₃—. In certain embodiments, L¹ is —(CH₂)₁₀—. In certain embodiments, L¹ is —(CH₂)₂₀—. In certain embodiments, L¹ is —(CH₂)₃₀—. In certain embodiments, L¹ is —CH₂CH(OR⁵)CH₂—. In certain embodiments, L¹ is —(CH₂)₂NR^{3c}—. In certain embodiments, L¹ is

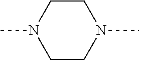

In certain embodiments, L¹ is

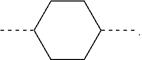

In certain embodiments, L¹ is

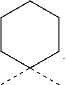

For instances of L which are asymmetric (e.g., —(CH₂)₃O—) it is understood that the present disclosure encompasses both possible orientations (e.g., —(CH₂)₃O— and —O(CH₂)₃—).

In certain embodiments, the ionizable lipid of Formula (I) is:

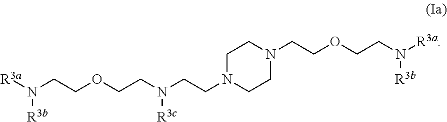

In certain embodiments, the ionizable lipid of Formula (I) is:

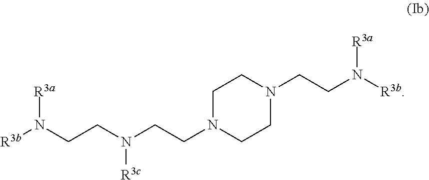

In certain embodiments, the ionizable lipid of Formula (I) is:

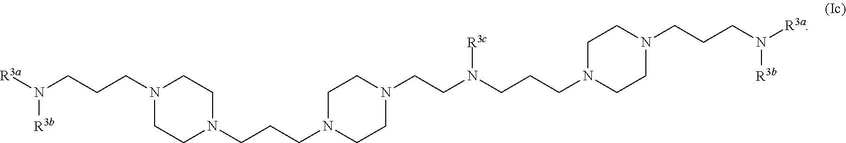

In certain embodiments, the ionizable lipid of Formula (I) is:

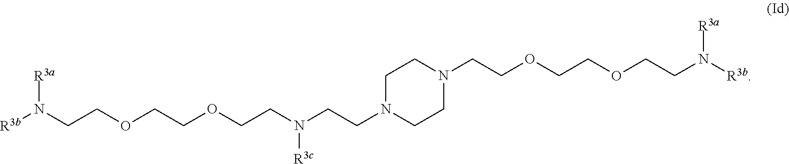

In certain embodiments, the ionizable lipid of Formula (I) is:

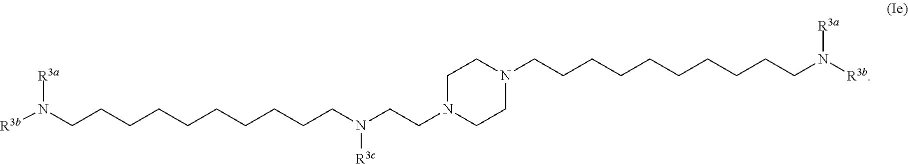

In certain embodiments, the ionizable lipid of Formula (I) is:

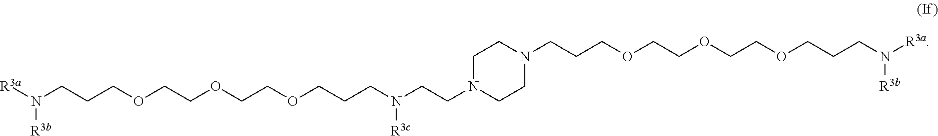

In certain embodiments, the ionizable lipid of Formula (I) is:

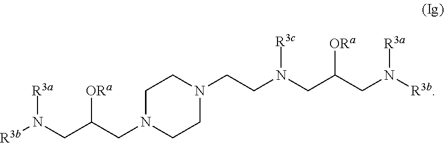

In certain embodiments, the ionizable lipid of Formula (I) is:

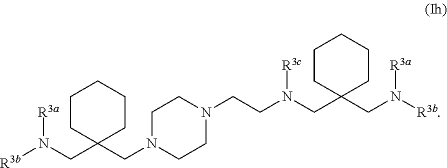

In certain embodiments, the ionizable lipid of Formula (I) is:

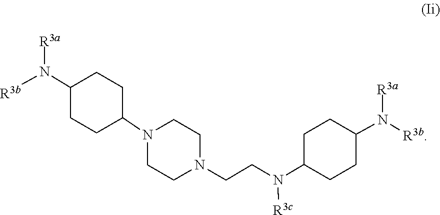

In certain embodiments, R^{3a} is H. In certain embodiments, R^{3a} is —CH₂CH(OH)(optionally substituted C₁-C₂₈ alkyl). In certain embodiments, R^{3a} is —CH₂CH(OH)(optionally substituted C₂-C₂₈ alkenyl). In certain embodiments, R^{3a} is —CH₂CH₂C(═O)O(optionally substituted C₁-C₂₈ alkyl). In certain embodiments, R^{3a} is —CH₂CH₂C(═O)NH(optionally substituted C₁-C₂₈ alkyl). In certain embodiments, R^{3b} is H. In certain embodiments, R^{3b} is —CH₂CH(OH)(optionally substituted C₁-C₂₈ alkyl). In certain embodiments, R^{3b} is —CH₂CH(OH)(optionally substituted C₂-C₂₈ alkenyl). In certain embodiments, R^{3b} is —CH₂CH₂C(═O)O(optionally substituted C₁-C₂₈ alkyl). In certain embodiments, R^{3b} is —CH₂CH₂C(═O)NH(optionally substituted C₁-C₂₈ alkyl). In certain embodiments, R^{3c} is H. In certain embodiments, R^{3c} is —CH₂CH(OH)(optionally substituted C₁-C₂₈ alkyl). In certain embodiments, R^{3c} is —CH₂CH(OH)(optionally substituted C₂-C₂₈ alkenyl). In certain embodiments, R^{3c} is —CH₂CH₂C(═O)O(optionally substituted C₁-C₂₈ alkyl). In certain embodiments, R^{3c} is —CH₂CH₂C(═O)NH(optionally substituted C₁-C₂₈ alkyl).

In certain embodiments, R^{3a} is —CH₂CH(OH)(CH₂)₉CH₃. In certain embodiments, R^{3a} is —CH₂CH(OH)(CH₂)₁₁CH₃. In certain embodiments, R^{3a} is —CH₂CH(OH)(CH₂)₁₃CH₃. In certain embodiments, R^{3b} is —CH₂CH(OH)(CH₂)₉CH₃. In certain embodiments, R^{3b} is —CH₂CH(OH)(CH₂)₁₁CH₃. In certain embodiments, R^{3b} is —CH₂CH(OH)(CH₂)₁₃CH₃. In certain embodiments, R^{3c} is —CH₂CH(OH)(CH₂)₉CH₃. In certain embodiments, R^{3c} is —CH₂CH(OH)(CH₂)₁₁CH₃. In certain embodiments, R^{3c} is —CH₂CH(OH)(CH₂)₁₃CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the ionizable lipid of Formula (I) is:

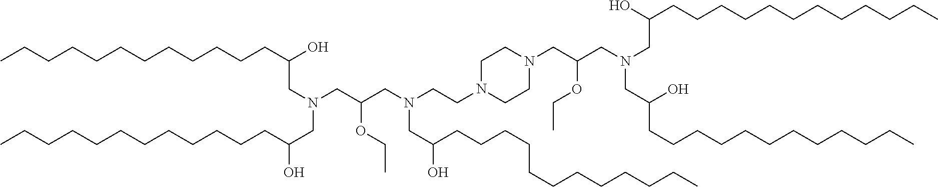 [SMILES: CCCCCCCCCCCCC(O)CN(CCN1CCN(CC(CN(CC(O)CCCCCCCCCCCC)CC(O)CCCCCCCCCCCC)OCC)CC1)CC(CN(CC(O)CCCCCCCCCCCC)CC(O)CCCCCCCCCCCC)OCC]

### 1,1′-((3-(4-(2-((3-(bis(2-hydroxytetradecyl)amino)-2-ethoxypropyl)(2-hydroxytetradecyl)amino)ethyl)piperazin-1-yl)-2-ethoxypropyl)azanediyl)bis(tetradecan-2-ol) (C14-490)

In certain embodiments, the ionizable lipid is SM-102:

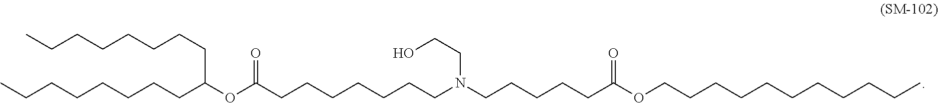 [SMILES: CCCCCCCCCCCOC(=O)CCCCCN(CCO)CCCCCCCC(=O)OC(CCCCCCCC)CCCCCCCC]

Ionizable Lipids and/or Cationic Lipids

The scope of ionizable lipids contemplated for use in the present disclosure is not limited to ionizable lipids of Formula (I). In the lipid nanoparticles of the disclosure, the cationic lipid or ionizable lipid may comprise, e.g., one or more of the following: (6Z,9Z,28Z,31Z)-heptatriaconta-6,9,28,31-tetraen-19-yl 4-(dimethylamino)butanoate (DLinMC3DMA), [(4-hydroxybutyl)azanediyl]di(hexane-6,1-diyl) bis(2-hexyldecanoate) (ALC-0315), heptadecan-9-yl 8-{(2-hydroxyethyl)[6-oxo-6-(undecyloxy)hexyl]amino}octanoate (SM-102), 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethylaminopropyl)-1,3]-dioxolane (D Lin-K-C3-D MA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-Nmethylpepiazino-[1,3]-dioxolane (DLin-K-MPZ), 2,2-dili-noleyl-4-dimethylaminomethyl-[1,3]-dioxolane (DLin-K-DMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (D Lin-MPZ), 3-(N,N-dilinoleylamino)-1,2-propanediol (D LinAP), 3-(N,N-dioleylamino)-1,2-propanedio (DOAP), 1,2-dilinoleyloxo-3-(2-N,N-dimethylamino)ethoxypropane (D Lin-EG-D MA), N,N-dioleyl-N,N-dimethylanrmonium chloride (DODAC), 1,2-dioleyloxy-N,N-dimethylaminopropane (DODMA), 1,2-distearyloxy-N,N-dimethylaminopropane (DSD MA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N, N-trimethylammonium chloride (DOTAP), 3-(N-(N′,N′dimethylaminoethane)-carbamoyl)cholesterol (DC-Chol), N-(1,2-dimyristyloxyprop-3-yl)-N,N-dimethyl-N-hydroxyethyl anrmonium bromide (DMRIE), 2,3-dioleyloxy-N-[2 (spermine-carboxamidoethyl]-N,N-dimethy 1-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof. The ionizable lipids are not limited to those recited herein, and can further include ionizable lipids known to those skilled in the art, or described in PCT Application No. PCT/US2020/056255 and/or PCT Application No. PCT/US2020/056252, the disclosures of which are herein incorporated by reference in its entirety.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Application Publication No. US 2011/0256175, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.Cl, DLin-TAP.Cl, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. US20060240554, the disclosure of which is herein incorporated by reference in its entirety for all purposes.

### Non-Cationic Lipid

In the nucleic acid-lipid particles of the present disclosure, the non-cationic lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S-NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (C₁₈), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Lipid Nanoparticles (LNPs)

In one aspect, the disclosure provides a lipid nanoparticle (LNP) composition. In certain embodiments, the LNP comprises at least one ionizable lipid. In certain embodiments, the LNP comprises at least one neutral lipid. In certain embodiments, the LNP comprises cholesterol lipid and/or a modified derivative thereof. In certain embodiments, the LNP comprises at least one polymer conjugated lipid and/or a modified derivative thereof. In certain embodiments, the LNP comprises a cell targeting polypeptide which specifically binds to a receptor overexpressed on the surface of a brain endothelial cell or neuron. In certain embodiments, the cell targeting domain is covalently conjugated to at least one component of the LNP.

In certain embodiments, the cell targeting polypeptide shares at least 85% sequence homology with RVG29 (SEQ ID NO:1), optionally wherein the cell targeting polypeptide specifically binds to a nicotinic acetylcholine receptor. In certain embodiments, the cell targeting polypeptide shares at least 85% sequence homology with T7 (SEQ ID NO:2). In certain embodiment, the cell targeting polypeptide specifically binds to a transferrin receptor. In certain embodiments, the cell targeting polypeptide shares at least 85% sequence homology with angiopep-2 (AP2) (SEQ ID NO:3). In certain embodiments, the cell targeting polypeptide specifically binds to low density lipoprotein receptor-related protein 1. In certain embodiments, the cell targeting polypeptide shares at least 85% sequence homology with mApoE (SEQ ID NO: 4). In certain embodiments, the cell targeting polypeptide specifically binds to a low density lipoprotein receptor (LDLR).

In certain embodiments, the at least one ionizable lipid comprises or consists essentially of SM-102:

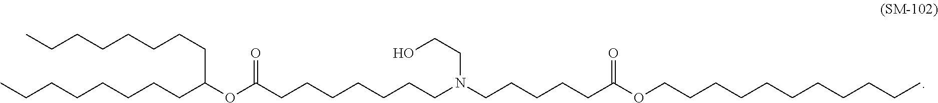 [SMILES: CCCCCCCCCCCOC(=O)CCCCCN(CCO)CCCCCCCC(=O)OC(CCCCCCCC)CCCCCCCC]

In certain embodiments, the at least one ionizable lipid comprises less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 mol % of the LNP. In certain embodiments, the at least one ionizable lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 mol % of the LNP. In certain embodiments, the at least one ionizable lipid comprises greater than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 mol % of the LNP.

In certain embodiments, the at least one ionizable lipid comprises less than about 50 mol % of the LNP. In certain embodiments, the at least one ionizable lipid comprises about 50 mol % of the LNP. In certain embodiments, the at least one ionizable lipid comprises greater than about 50 mol % of the LNP.

In certain embodiments, the neutral lipid is distearoylphosphatidylcholine (DSPC). In certain embodiments, the neutral lipid is dioleoylphosphatidylethanolamine (DOPE).

In certain embodiments, the at least one neutral lipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the at least one neutral lipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the at least one neutral lipid comprises greater than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one neutral lipid comprises less than about 10 mol % of the LNP. In certain embodiments, the at least one neutral lipid comprises about 10 mol % of the LNP. In certain embodiments, the at least one neutral lipid comprises greater than about 10 mol % of the LNP.

In certain embodiments, the at least one cholesterol lipid and/or modified derivative thereof comprises or consists essentially of cholesterol.

In certain embodiments, the cholesterol and/or modified derivative thereof comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP. In certain embodiments, the cholesterol and/or modified derivative thereof comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP. In certain embodiments, the cholesterol and/or modified derivative thereof comprises greater than about 5, 6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the cholesterol and/or modified derivative thereof comprises less than about 38.5 mol % of the LNP. In certain embodiments, the cholesterol and/or modified derivative thereof comprises about 38.5 mol % of the LNP. In certain embodiments, the cholesterol and/or modified derivative thereof comprises greater than about 38.5 mol % of the LNP.

In certain embodiments, the at least one polymer conjugated lipid and/or modified derivative thereof comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.2, 5.4, 5.5, 5.6, 5.7, 5.8, 5.9, 6.0, 6.1, 6.2, 6.2, 6.4, 6.5, 6.6, 6.7, 6.8, 6.9, 7.0, 7.1, 7.2, 7.2, 7.4, 7.5, 7.6, 7.7, 7.8, 7.9, 8.0, 8.1, 8.2, 8.2, 8.4, 8.5, 8.6, 8.7, 8.8, 8.9, 9.0, 9.1, 9.2, 9.2, 9.4, 9.5, 9.6, 9.7, 9.8, 9.9, 10.0, 10.1, 10.2, 10.2, 10.4, 10.5, 10.6, 10.7, 10.8, 10.9, 11.0, 11.1, 11.2, 11.2, 11.4, 11.5, 11.6, 11.7, 11.8, 11.9, 12.0, 12.1, 12.2, 12.3, 12.4, or about 12.5 mol % of the LNP.

In certain embodiments, the at least one polymer conjugated lipid and/or modified derivative thereof comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.2, 5.4, 5.5, 5.6, 5.7, 5.8, 5.9, 6.0, 6.1, 6.2, 6.2, 6.4, 6.5, 6.6, 6.7, 6.8, 6.9, 7.0, 7.1, 7.2, 7.2, 7.4, 7.5, 7.6, 7.7, 7.8, 7.9, 8.0, 8.1, 8.2, 8.2, 8.4, 8.5, 8.6, 8.7, 8.8, 8.9, 9.0, 9.1, 9.2, 9.2, 9.4, 9.5, 9.6, 9.7, 9.8, 9.9, 10.0, 10.1, 10.2, 10.2, 10.4, 10.5, 10.6, 10.7, 10.8, 10.9, 11.0, 11.1, 11.2, 11.2, 11.4, 11.5, 11.6, 11.7, 11.8, 11.9, 12.0, 12.1, 12.2, 12.3, 12.4, or about 12.5 mol % of the LNP.

In certain embodiments, the at least one polymer conjugated lipid and/or modified derivative thereof comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.2, 5.4, 5.5, 5.6, 5.7, 5.8, 5.9, 6.0, 6.1, 6.2, 6.2, 6.4, 6.5, 6.6, 6.7, 6.8, 6.9, 7.0, 7.1, 7.2, 7.2, 7.4, 7.5, 7.6, 7.7, 7.8, 7.9, 8.0, 8.1, 8.2, 8.2, 8.4, 8.5, 8.6, 8.7, 8.8, 8.9, 9.0, 9.1, 9.2, 9.2, 9.4, 9.5, 9.6, 9.7, 9.8, 9.9, 10.0, 10.1, 10.2, 10.2, 10.4, 10.5, 10.6, 10.7, 10.8, 10.9, 11.0, 11.1, 11.2, 11.2, 11.4, 11.5, 11.6, 11.7, 11.8, 11.9, 12.0, 12.1, 12.2, 12.3, 12.4, or about 12.5 mol % of the LNP.

In certain embodiments, the at least one polymer conjugated lipid and/or modified derivative thereof comprises less than about 1.5 mol % of the LNP. In certain embodiments, the at least one polymer conjugated lipid and/or modified derivative thereof comprises about 1.5 mol % of the LNP. In certain embodiments, the at least one polymer conjugated lipid and/or modified derivative thereof comprises greater than about 1.5 mol % of the LNP.

In certain embodiments, the at least one polymer conjugated lipid and/or modified derivative thereof comprises 1,2-distearoyl-sn-glycero-3-phosphoethanolamine polyethylene glycol 2000 (DSPE-PEG-2000).

In certain embodiments, the cell targeting polypeptide which specifically binds to a receptor overexpressed on the surface of a brain endothelial cell or neuron is covalently conjugated is the modified derivative of the polymer conjugated lipid. In certain embodiments, the covalent conjugation comprises a covalent bond forming reaction selected from the group consisting of a [1,4]-conjugate addition (i.e., Michael addition), [4+2]cycloaddition, [3+2]dipolar cycloaddition, nucleophilic addition, transition metal-catalyzed cross-coupling reaction, carbonyl condensation reaction, and reductive amination. In certain embodiments, the covalent conjugation reaction comprises a [1,4]-conjugate addition reaction (i.e., Michael addition). In certain embodiments, the [1,4]-conjugate addition occurs between a maleimide moiety of the modified derivative of the polymer conjugated and a cysteine thiol on the polypeptide.

In certain embodiments, the modified derivative of the polymer conjugated lipid is a compound of Formula (II), or a salt, solvate, stereoisomer, or isotopologue thereof:

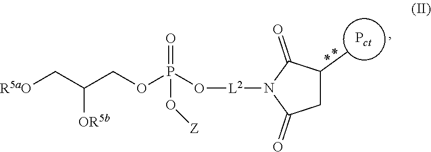

wherein:
R^{5a} and R^{5b} are each independently selected form the group consisting of —C(═O)(optionally substituted C₁-C₂₈ alkyl), —C(═O)(optionally substituted C₂-C₂₈ alkenyl), —C(═O)(optionally substituted C₂-C₂₈ alkynyl), optionally substituted C₁-C₂₈ alkyl, optionally substituted C₂-C₂₈ alkenyl, and optionally substituted C₂-C₂₈ alkynyl;
Z is a monovalent cation;
L² comprises n units of

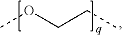

o units of

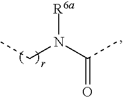

and p units of

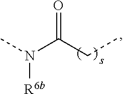

wherein each [symbol] in L² is a C—O or C—N bond;
P_{ct} is a polypeptide which shares at least 85% sequence homology with a polypeptide selected from the group consisting of RVG29 (SEQ ID NO:1), T7 (SEQ ID NO:2), AP2 (SEQ ID NO:3), and mApoE (SEQ ID NO:4),
   wherein [symbol] is C—S bond;
R^{6a} and R^{6b} are each independently selected from the group consisting of H and C₁-C₆ alkyl;
n, o, and p are each independently 1, 2, 3, 4, or 5;
q is an integer ranging from 1 to 100; and
r and s are each independently 1, 2, 3, 4, 5, 6, 7, 8, 9, or 10.

In certain embodiments, Z is NH₄⁺.

In certain embodiments, L² is

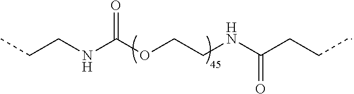

In certain embodiments, compound of formula (II) is:

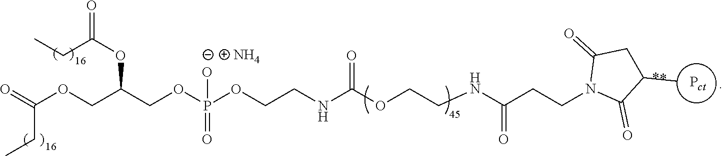

In certain embodiments, (d) comprises the polymer conjugated lipid and the compound of formula (II), wherein the polymer conjugated lipid and the compound of formula (II) have a molar ratio of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10. In certain embodiments, the molar ratio of polymer conjugated lipid and modified derivative of the conjugated lipid further conjugated to a maleimide moiety is about 2:1.

In certain embodiments, the LNP further comprises at least one cargo selected from the group consisting of a nucleic acid molecule and a therapeutic agent.

In certain embodiments, the therapeutic agent is at least one selected from the group consisting of a small molecule, a protein, and an antibody.

In certain embodiments, the LNP comprises a nucleic acid molecule.

In certain embodiments, the nucleic acid molecule is a DNA molecule or an RNA molecule.

In certain embodiments, the nucleic acid molecule is selected from the group consisting of cDNA, mRNA, miRNA, siRNA, modified RNA, antagomir, antisense molecule, and a targeted nucleic acid, or any combination thereof.

In certain embodiments, the nucleic acid molecule encodes a chimeric antigen receptor (CAR).

In certain embodiments, the CAR is specific for binding to a surface antigen of a pathogenic cell.

In certain embodiments, the nucleic acid molecule encodes mRNA. In certain embodiments, the nucleic acid molecule encodes sgRNA. In certain embodiments, the nucleic acid molecule encodes mRNA and sgRNA. In certain embodiments, the mRNA encodes a therapeutic protein. In certain embodiments, the therapeutic protein is a CRISPR-associated protein. In certain embodiments, the CRISPR-associated protein is CRISPR-associated protein 9 (Cas9).

In certain embodiments, the therapeutic agent is a CRISPR-associated protein. In certain embodiments, the CRISPR-associated protein is CRISPR-associated protein 9 (Cas9).

### LNP Cargo

### Anti-Cancer Agents

In one embodiment, the at least one additional agent is an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but are not limited to, chemotherapeutic agents, antiproliferative agents, anti-tumor agents, checkpoint inhibitors, and anti-angiogenic agents. For example, in one embodiment, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acids

In certain embodiments, the invention includes an ionizable LNP molecule formulated for targeted in vivo T cell delivery comprising or encapsulating one or more nucleic acid molecule. In certain embodiments, the nucleic acid molecule is a mRNA molecule. In certain embodiments, the mRNA molecule encodes a CAR. In certain embodiments, the nucleoside-modified mRNA molecule encodes a CAR. In certain embodiments, the invention includes a nucleoside-modified mRNA molecule encoding an adjuvant.

The nucleotide sequences encoding an CAR, as described herein, can alternatively comprise sequence variations with respect to the original nucleotide sequences, for example, substitutions, insertions and/or deletions of one or more nucleotides, with the condition that the resulting polynucleotide encodes a polypeptide according to the invention. Therefore, the scope of the present invention includes nucleotide sequences that are substantially homologous to the nucleotide sequences recited herein and encode an antigen or antigen binding molecule or adjuvant of interest.

Further, the scope of the invention includes nucleotide sequences that encode amino acid sequences that are substantially homologous to the amino acid sequences recited herein and preserve the immunogenic function of the original amino acid sequence.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In certain embodiments, the invention relates to a construct, comprising a nucleotide sequence encoding a CAR. In certain embodiments, the construct comprises a plurality of nucleotide sequences encoding a plurality of antigens. For example, in certain embodiments, the construct encodes 1 or more, 2 or more, 5 or more, 10 or more, 15 or more, or 20 or more antigens. In certain embodiments, the invention relates to a construct, comprising a nucleotide sequence encoding an adjuvant. In certain embodiments, the construct comprises a first nucleotide sequence encoding a CAR and a second nucleotide sequence encoding an adjuvant.

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding a CAR; and a second construct, comprising a nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Polypeptide Therapeutic Agents

In other related aspects, the therapeutic agent includes an isolated peptide that modulates a target. For example, In certain embodiments, the peptide of the invention inhibits or activates a target directly by binding to the target thereby modulating the normal functional activity of the target. In certain embodiments, the peptide of the invention modulates the target by competing with endogenous proteins. In certain embodiments, the peptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### CAR Agents

In certain embodiments, the mRNA molecule of the invention encodes a chimeric antigen receptor (CAR). In certain embodiments, the CAR comprises an antigen binding domain. In certain embodiments, the antigen binding domain is a targeting domain, wherein the targeting domain directs the T cell expressing the CAR to a specific cell or tissue of interest. For example, In certain embodiments, the targeting domain comprises an antibody, antibody fragment, or peptide that specifically binds to an expressed on a pathogenic organism or a tumor cell thereby directing the T cell expressing the CAR to a cell or tissue expressing the antigen.

In certain embodiments, the invention relates to an immune cell targeted LNP comprising an agent, wherein the agent comprises a nucleic acid sequence encoding a chimeric antigen receptor (CAR). In certain embodiments, agent comprises an mRNA molecule encoding a CAR. In certain embodiments, the agent comprises a modified nucleoside mRNA molecule encoding a CAR.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have 15 indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2RP.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is an scFv specific for binding to a surface antigen of a target cell of interest (e.g., a pathogen or tumor cell.)

### Combinations

In certain embodiments, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Methods

In one aspect, the disclosure provides a method of treating, preventing, and/or ameliorating a disease or disorder in a subject. In certain embodiments, the method comprises administering to the subject the LNP of the disclosure or the pharmaceutical composition of the disclosure.

In certain embodiments, the disease or disorder is associated with the brain. In certain embodiments, the disease or disorder is a neurological disease or disorder.

In another aspect, the disclosure provides a method of delivering at least one cargo molecule to a brain endothelial cell or neuronal cell of a subject. In certain embodiments, the method comprises administering to the subject the LNP of the disclosure or the pharmaceutical composition of the disclosure.

In certain embodiments, the subject is a mammal. In certain embodiments, the mammal is a human.

### Pharmaceutical Compositions

In another aspect, the present disclosure provides a pharmaceutical composition comprising the lipid nanoparticle (LNP) of the present disclosure and at least one pharmaceutically acceptable carrier. In certain embodiments, the composition further comprises at least one adjuvant. In certain embodiments, the composition is a vaccine.

Such a pharmaceutical composition may consist of at least one composition of the invention, in a form suitable for administration to a subject, or the pharmaceutical composition may comprise at least one composition, and one or more pharmaceutically acceptable carriers, one or more additional ingredients, or any combinations of these. At least one composition of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXAMPLES

Various embodiments of the present application can be better understood by reference to the following Examples which are offered by way of illustration. The scope of the present application is not limited to the Examples given herein.

### Materials and Methods

### Materials and Reagents

SM-102 was purchased from Cayman Chemical (USA). 1,2-distearoyl-sn-glycero-3-phosphocholine (DSPC), 1,2-dimyristoyl-rac-glycero-3-methoxypolyethylene glycol-2000 (DMG-PEG2 k), and 1,2-distearoyl-sn-glycero-3-phosphoethanolamine-N-[maleimide(polyethylene glycol)-2000](ammonium salt) (DSPE-PEG2 k-maleimide) were purchased from Avanti Polar Lipids (USA). Cholesterol, hCMEC/D3 cells, EndoGRO-MV Complete Culture Media Kit, Fibroblast Growth Factor-basic protein (human recombinant), Collagen Type I rat tail, Accumax, and sterile-filtered human serum from human male AB plasma were purchased from Sigma-Aldrich (USA). CleanCap® Firefly Luciferase mRNA and CleanCap® mCherry mRNA with full N1-methylpseudouridine substitutions were purchased from TriLink BioTechnologies (USA). RVG29 (YTIWMPENPRPGTPCDIFTNSRGKRASNGC) (SEQ ID NO:1), T7 (CHAIYPRH) (SEQ ID NO:2), angiopep-2 (TFFYGGSRGKRNNFKTEEYC) (SEQ ID NO:3), and mApoE (RLLRKRLKRLGWC) (SEQ ID NO:5) peptides with >98% purity were purchased from Biomatik (USA). Triton X-100 was purchased from Alfa Aesar (USA). Quant-it™ RiboGreen RNA Assay Kit, CBQCA Protein Quantitation Kit, DiR, Hoechst 33342, LysoTracker Deep Red, Calcein AM, Total Exosome Isolation Reagent, UltraPure™ 0.5M EDTA pH 8.0, and mCherry Monoclonal Antibody (16D7) Alexa Fluor™ 594 (M11240) were purchased from ThermoFisher Scientific (USA). FluoProdige Protein Quantification Assay Kit was purchased from OZ Biosciences (USA). SH-SY5Y cells were purchased from the American Type Culture Collection (USA). Penicillin-Streptomycin, Dulbecco's Modified Eagle Medium (DMEM), heat-inactivated fetal bovine serum (FBS), GlutaMAX™, Trypsin-EDTA (0.25%), and Opti-MEM™ were purchased from Gibco (USA). HTS Transwell® 96-well permeable supports (1 m pore size, PET membrane) were purchased from Corning (USA). Reporter Lysis 5× Buffer, Luciferase Assay Substrate and Buffer, and CellTiter-Glo® Luminescent Cell Viability Assay were purchased from Promega (USA). D-Luciferin Potassium Salt was purchased from Regis Technologies (USA). Liberase™ TL Research Grade and DNase I recombinant were purchased from Roche (Switzerland). ACK Lysing Buffer was purchased from Quality Biological (USA). Zombie UV™ Fixable Viability Kit, True-Nuclear™ Transcription Factor Buffer Set, TruStain FcX™ PLUS (anti-mouse CD16/32) Antibody (156604), PE/Cyanine7 anti-mouse CD31 Antibody (102523), and Brilliant Violet 421™ anti-GFAP Antibody (644710) were purchased from BioLegend (USA). Recombinant Alexa Fluor® 647 Anti-NeuN Antibody (ab190565) was purchased from Abcam (USA).

### LNP Formulation and Characterization

LNPs were formulated at a 10:1 weight ratio of ionizable lipid to mRNA. Ionizable lipid, phospholipid, cholesterol, lipid-PEG, and lipid-PEG-maleimide were combined at various molar ratios into an ethanol phase (Table 1). mRNA was dissolved in 10 mM citrate buffer (pH 3) to produce an aqueous phase. The ethanol and aqueous phases were combined at 1:3 volumetric and flow rate ratios in a microfluidic device using syringe pumps to produce LNPs. LNPs were dialyzed in 20K MWCO cassettes against 1×PBS for 2 h to remove excess components and allow for buffer exchange, and sterile filtered with 0.22 m filters. To make peptide-conjugated LNPs, peptides were added to LNPs at a peptide:lipid-PEG-maleimide molar ratio of 2:1 and shaken at 300 rpm and room temperature for 1 h, followed by overnight incubation at 4° C. Peptide-conjugated LNPs were then dialyzed in 20K MWCO cassettes against 1×PBS for 2 h to remove unreacted peptides. LNPs were stored at 4° C. until use. For LNPs loaded with DiR dye, DiR was added to the LNPs immediately following microfluidic formulation such that excess dye could be removed during subsequent dialysis.

TABLE 1
Formulation of exemplary LNPs of the disclosure

| LNP | Lipid-PEG-mal (Mal) | Molar Ratio (IL/PL/Ch/PEG/Mal) |
|---|---|---|
| 0% | — | 50/10/38.5/1.5/0 |
| 10% | DSPE-PEG2k-mal | 50/10/38.5/1.35/0.15 |
| 30% | DSPE-PEG2k-mal | 50/10/38.5/1.05/0.45 |
| 50% | DSPE-PEG2k-mal | 50/10/38.5/0.75/0.75 |

Ionizable lipid (IL): SM-102; Phospholipid (PL): DSPC; Cholesterol (Ch): Cholesterol; Lipid-PEG (PEG): DMG-PEG2k.

To measure size (Z-average diameter) and polydispersity index (PDI), LNPs were diluted 6-fold in 1×PBS and added to a 384-well plate for dynamic light scattering measurements using a DynaPro® Plate Reader III (Wyatt Technology, USA). To measure zeta potential, 10 μL of LNPs were combined with 790 μL of deionized water, added to a DTS1070 cuvette (Malvern Panalytical, UK), and read on a Zetasizer Nano (Malvern Panalytical). To measure mRNA encapsulation efficiency and concentration, the Quant-it™ RiboGreen RNA Assay Kit was used as previously described.

To quantify peptide concentration, two commercially available kits were used. The FluoProdige Protein Quantification Assay Kit was used per the manufacturer's instructions. Briefly, 50 μL of LNPs or BSA standard in PBS were combined with 50 μL of FluoProdige Dye working solution. After 15 minutes of incubation at room temperature, fluorescence was read at an excitation/emission wavelength of 518/605 nm on a plate reader (Tecan, USA). The CBQCA Protein Quantitation Kit was also used per the manufacturer's instructions. LNPs and BSA standards were prepared in 0.1 M sodium borate containing 0.1% Triton X-100 and combined with 20 mM KCN and 40 mM ATTO-TAG CBQCA reagent to a total reaction volume of 100 μL at the ratios specified in the manufacturer's instructions. After 1 h of incubation at room temperature on a plate shaker at 300 rpm, fluorescence was read at an excitation/emission wavelength of 465/550 nm on a plate reader. For both assays, LNP peptide concentration was calculated by subtracting the base fluorescence from an LNP with no peptide and then converting the fluorescence to concentration using a BSA standard curve.

### Cell Culture

hCMEC/D3 cells were grown in flasks coated with collagen, cultured in EndoGRO-MV Complete Culture Media supplemented with 1 ng/mL Fibroblast Growth Factor-basic and 1% penicillin streptomycin, and detached with Accumax. SH-SY5Y cells were cultured in DMEM supplemented with 10% heat-inactivated FBS, 1× GlutaMAX™, and 1% penicillin streptomycin, and detached with trypsin-EDTA. Both cell lines were maintained at 37° C. and 5% CO₂, passaged at 80% confluency, and not used past passage 15.

### In Vitro LNP Delivery

Cells were seeded at 20,000 cells/cm² in 100 μL of serum-supplemented growth media or serum-free Opti-MEM™ in 96-well plates (which were coated with collagen for hCMEC/D3 cells) and treated with LNPs the next day at 60 ng mRNA/20,000 cells for 24 h, unless specified otherwise. To measure cell viability after LNP treatment, 100 μL of CellTiter-Glo was added into the media of each well. After 5 minutes of incubation at room temperature on a plate shaker at 300 rpm, luminescent signal was measured using a plate reader. To measure luciferase expression, media was removed from each well, cells were lysed with 50 μL of 1× lysis buffer, and 100 μL of luciferase assay substrate was added. After 5 minutes of incubation at room temperature on a plate shaker at 300 rpm, luminescent signal was measured using a plate reader. For the human serum incubation study, LNPs were incubated in 40% (v/v) human serum for 30 minutes at 37° C. with shaking at 300 rpm prior to treatment.

For the cellular trafficking study, cells were treated with DiR-loaded LNPs for 2 h, following which media was replaced and cells were incubated for up to 24 h total. Cells were then stained with Hoechst 33342 at 10 g/mL and LysoTracker Deep Red at 75 nM for 30 minutes at 37° C. and imaged using a widefield fluorescence microscope. To image cells treated with mCherry mRNA LNPs, cells were incubated with Calcein AM at 2 M for 15 minutes at 37° C. to stain living cells and imaged using a widefield fluorescence microscope.

### Transwell Study

hCMEC/D3 monolayers were grown in 96-well transwell inserts as previously described. Briefly, transwell inserts were coated with 10 g/cm² collagen and seeded with hCMEC/D3 cells at 30,000 cells/cm² in 75 μL of media on Day 0. Media was changed on Days 3 and 5. On Day 6, complete monolayer formation was confirmed with live/dead imaging. The monolayers were treated with LNPs at a dose of 60 ng mRNA/20,000 cells in 75 μL of media. The transwell inserts were then moved onto a 96-well plate containing SH-SY5Y cells with 235 μL of media in each well and incubated for 24 h.

To measure luciferase expression in hCMEC/D3 cells, the transwell inserts were moved to a new plate, media was removed from each transwell insert, cells were lysed with 25 μL of 1% Triton X-100 in PBS, and 50 μL of luciferase assay substrate was added. To measure luciferase expression in SH-SY5Y cells, media was removed from each well, cells were lysed with 50 μL of 1× lysis buffer, and 100 μL of luciferase assay substrate was added. After 5 minutes of incubation at room temperature on a plate shaker at 300 rpm, luminescent signal was measured using a plate reader.

### Exosome Isolation Study

hCMEC/D3 cells were seeded at 20,000 cells/cm² in 100 μL of media in collagen-coated 96-well plates and treated with LNPs the next day at 60 ng mRNA/20,000 cells. After 2 h, the media was replaced with fresh media to remove LNPs that were not endocytosed. After an additional 22 h, media was pooled from replicate wells for each treatment group, and exosomes were isolated using Total Exosome Isolation Reagent per the manufacturer's instructions. Luciferase expression in the remaining hCMEC/D3 cells was measured as described above. After the overnight exosome isolation, the exosomes were resuspended in PBS and equally split between replicate wells of SH-SY5Y cells grown in a 96-well plate. After 24 h of treatment, luciferase expression in the SH-SY5Y cells was measured as described above.

### In Vivo Biodistribution Studies

For luciferase mRNA LNPs, healthy female adult C57BL/6 mice (Jackson Laboratory, USA) were injected via the lateral tail vein with PBS or mRNALNPs at a dose of 0.3 mg/kg. After 6 h, mice were given an intraperitoneal injection of 150 μL of 30 mg/mL D-luciferin potassium salt in PBS. After 5 minutes, mice were euthanized and dissected to collect organs.

Organs were then imaged for luminescence using an in vivo imaging system (IVIS) (PerkinElmer, USA), and the luminescent signal was quantified using the Living Image software (PerkinElmer). For mCherry mRNA LNPs, healthy female adult C57BL/6 mice were injected via the lateral tail vein with PBS or mRNA LNPs at a dose of 0.3 mg/kg. After 12 h, mice were euthanized and dissected to collect organs.

### Brain Flow Cytometry

To prepare a single cell suspension, brains were first mechanically dissociated in ice-cold HBSS. Iris scissors were used to cut the tissue until pieces were small enough to be pipette-dissociated using a 1000 μL pipette tip. The suspension was then pipette dissociated until no resistance was detected. After centrifugation, pellets were resuspended in 2 mL of enzymatic digestion buffer containing Liberase™ at 2 U/mL and DNase I at 100 U/mL in HBSS and incubated for 30 minutes at 37° C. with shaking. The suspension was passed through a 100 m cell strainer, centrifuged, resuspended in 5 mL of ACK lysis buffer to remove red blood cells, centrifuged again, and resuspended in PBS with 2 mM EDTA.

Samples were then divided into tubes containing ~3 million cells each for staining. Cells were stained with 2 L of Zombie UV™ for 15 minutes at 4° C., washed with PBS with 2 mM EDTA, and blocked using a solution containing TruStain FcX™ antibody at 7.5 g/mL for 10 minutes at 4° C. Cells were stained for surface markers with 1 g of CD31 antibody for 30 minutes at 4° C., washed, and fixed and permeabilized using the True-Nuclear™ Transcription Factor Buffer Set. Cells were stained for intracellular and intranuclear markers with 1 g of GFAP antibody, 1 g of NeuN antibody, and 1 g of mCherry antibody for 30 minutes at room temperature, washed, resuspended in PBS with 2 mM EDTA, and moved to flow tubes.

### Statistical Analyses

All statistical analyses were conducted using GraphPad Prism. Significance was determined using one-way ANOVAs with multiple comparisons tests.

### Example 1: Functionalizing mRNA LNPs with Brain-Targeting Peptides

Efforts to determine if the RVG29, T7, AP2, and mApoE peptides could be stably conjugated to mRNA LNPs were first investigated. For the untargeted LNP, the SM-102 formulation was selected, which has demonstrated clinical success in Moderna's COVID-19 mRNA vaccine with intramuscular administration and was previously shown to enable moderate brain transfection after systemic administration. To formulate pLNPs, a fifth lipid component, DSPE-PEG-maleimide (lipid-PEG-mal), was incorporated, which permits covalent conjugation to a terminal cysteine on the peptide sequences via a thiol-maleimide reaction. Since other groups have shown that the surface density of targeting ligands affects delivery across the BBB in vitro and in vivo, pLNPs were formulated with varying amounts of lipid-PEG-mal, substituting it for 10%, 30%, or 50% of the original 1.5 mol % lipid-PEG (Table 1).

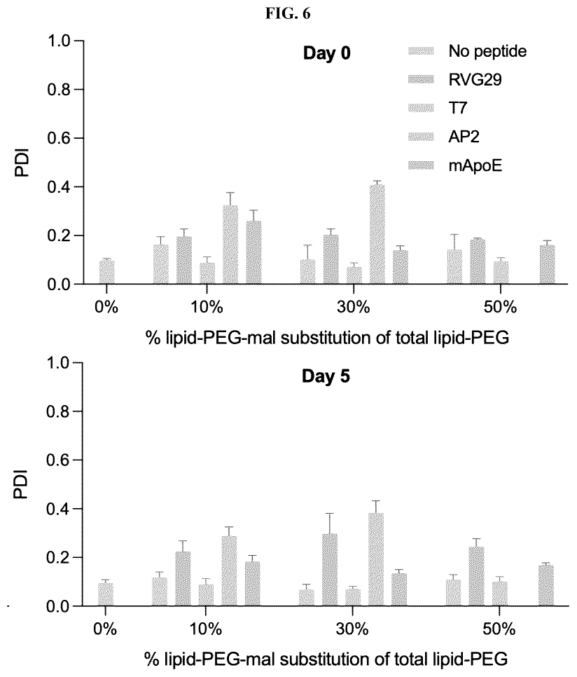

LNPs were formulated with luciferase mRNA via microfluidic mixing, then incubated with peptides post-formulation for click chemistry-mediated conjugation (FIG. 1). Overall, higher lipid-PEG-mal substitutions led to larger LNP size increases post-conjugation (FIG. 1C). Functionalization with RVG29 and AP2 led to the largest increase in size compared to untargeted controls, which was expected as these are the two largest peptides. T7 led to the smallest change in size, which was expected since it is the smallest peptide. The sizes of all pLNPs were stable 5 days post-formulation (FIG. 1C). Untargeted LNPs were monodisperse with a polydispersity index (PDI) of ~0.1, which was maintained for T7 LNPs at all substitutions (FIG. 6). RVG29 and mApoE LNPs had PDIs of ~0.2-0.3, while AP2 LNPs were more polydisperse with PDIs of ~0.3-0.4. The zeta potential of pLNPs was also assessed to investigate changes in surface charge following peptide functionalization (FIG. 1D). Notably, LNPs became less anionic with higher amounts of mApoE. This is likely due to the abundance of lysine and arginine in the mApoE sequence, both of which bear positive charges at neutral pH.

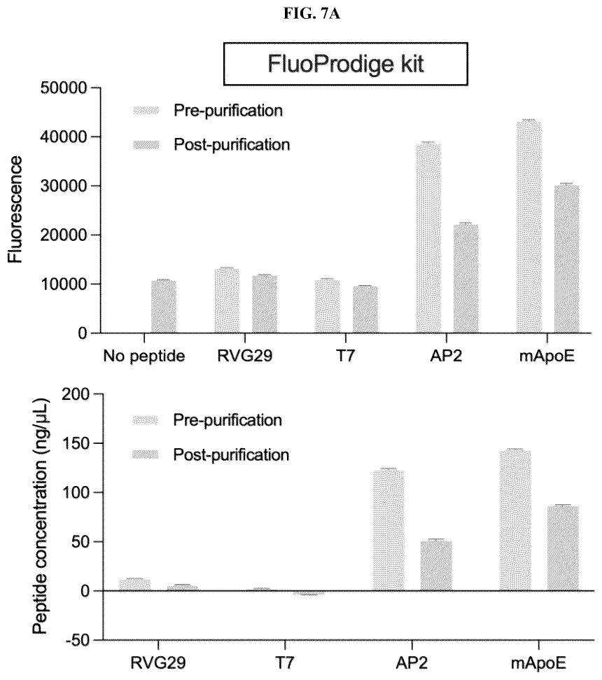

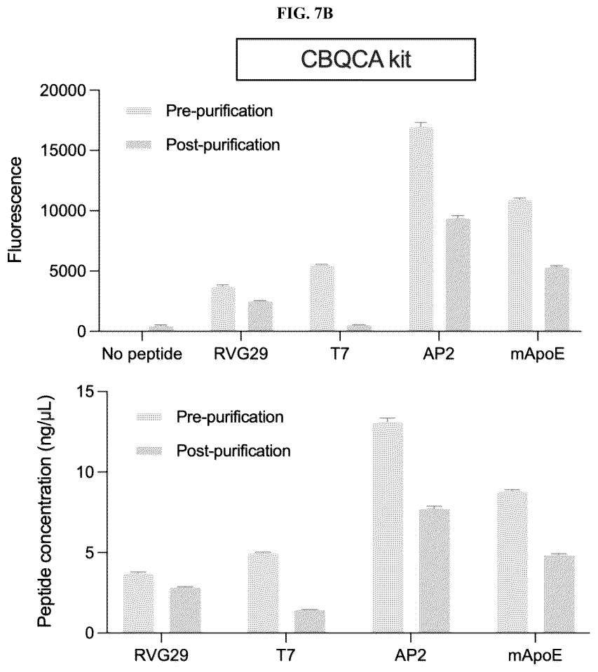

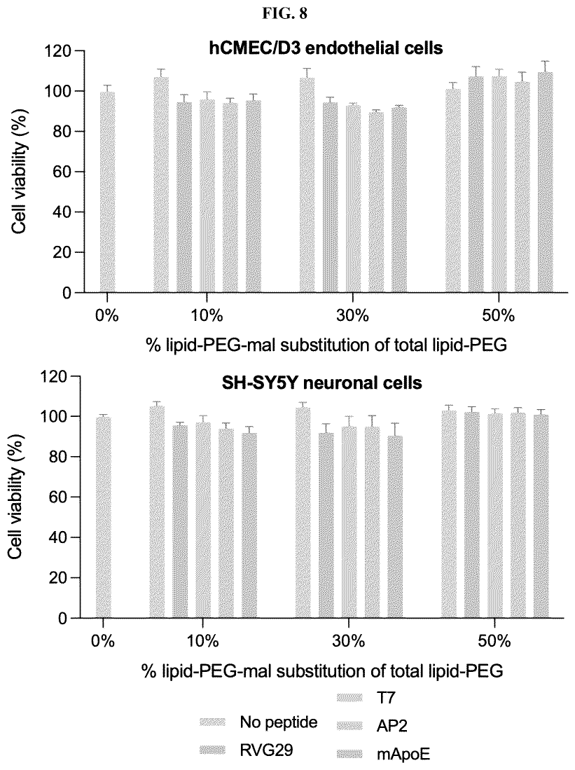

Peptide conjugation was confirmed using two fluorometric protein quantification kits. The FluoProdige kit uses a reagent which binds to lysine, arginine, and histidine. While this kit was able to show the relative difference in peptide concentration pre- and post-purification of excess peptides, the high base fluorescence from the untargeted LNP made interpretation of the peptide concentrations difficult (FIG. 7A). This problem was circumvented with the CBQCA kit, which uses a reagent that binds to primary amines and is optimized for protein detection in the presence of lipids and detergents. Using this kit, the untargeted LNP exhibited very low base fluorescence, and the pLNPs displayed peptide concentration values which more closely aligned with their expected yield (FIG. 7B). Overall, both kits demonstrate that purification reduces the peptide concentration by approximately half, which was expected given the conjugation reaction was set up at a 2:1 molar ratio of peptide:lipid-PEG-mal and suggests that most of the lipid-PEG-mal molecules are functionalized with peptide. Finally, it was confirmed that all pLNPs maintained high mRNA encapsulation efficiencies (FIG. 1E) and were not cytotoxic to hCMEC/D3 brain endothelial or SH-SY5Y neuronal cells (FIG. 8).

### Example 2: Peptide Targeting Improves mRNA Transfection of Brain Endothelial and Neuronal Cells

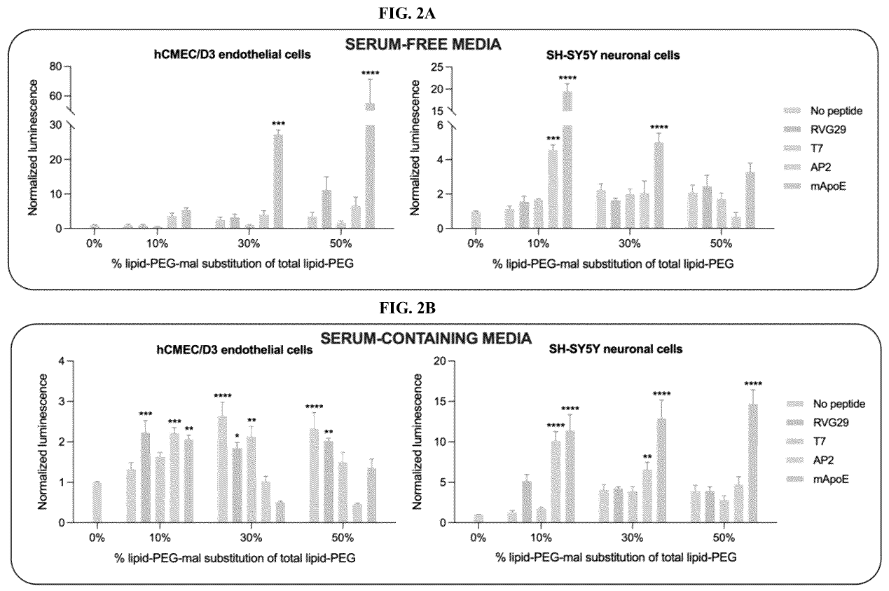

The potential of peptide functionalization to improve LNP-mediated luciferase mRNA transfection in vitro was then assessed. Transfection efficiency was first evaluated in serum-free media (FIG. 2A). In brain endothelial cells, mApoE improved transfection at all lipid-PEG-mal substitutions compared to untargeted 0% substitution LNPs, with 50% substitution mediating a significant ~60-fold improvement. RVG29 and AP2 at 50% substitution also improved transfection, whereas T7 did not at any substitution tested. In neuronal cells, mApoE LNPs were again the top performing formulations, improving transfection at all substitutions compared to 0% substitution LNPs, with 10% substitution mediating a significant ~20-fold improvement. AP2 at 10% substitution improved transfection by ~5-fold.

While the serum-free media condition was helpful in assessing the efficacy of peptide targeting, it does not account for the protein corona that forms around nanoparticles in physiological conditions, which may prevent targeting ligands from binding to their receptors. Thus, the transfection assays were repeated in media supplemented with 10% fetal bovine serum (FBS) (FIG. 2B). In brain endothelial cells, the drastic mApoE-induced improvement in transfection over untargeted LNPs seen in the serum-free conditions was no longer observed. Rather, LNPs with 30% and 50% lipid-PEG-mal substitutions without peptides exhibited a significant transfection increase compared to 0% substitution LNPs. The free maleimide groups on these LNPs may be reacting with free thiols on serum proteins, leading to improved cellular uptake. In neuronal cells, mApoE LNPs appeared less susceptible to serum-induced changes in transfection, with all substitutions mediating significant ~10- to 15-fold improvements compared to 0% substitution LNPs. Interestingly, AP2 LNPs in serum-containing media demonstrated greater transfection improvements over untargeted LNPs compared to the serum-free media, with all substitutions mediating ~5- to 10-fold improvements compared to 0% substitution LNPs. Similarly, RVG29 LNPs also facilitated greater transfection improvements over untargeted LNPs in serum-containing media, with all substitutions mediating ~5-fold improvements.

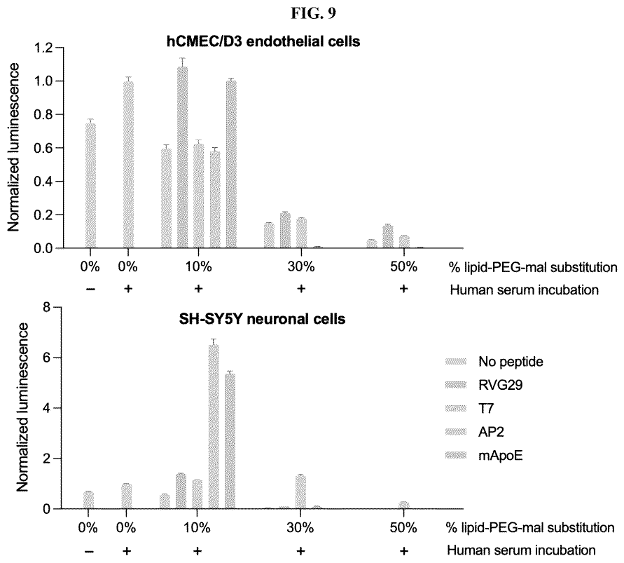

It was also assessed whether these trends would hold in more physiologically relevant conditions by incubating LNPs in 40% human serum prior to treatment (FIG. 9). In brain endothelial cells, RVG29 and mApoE LNPs at 10% lipid-PEG-mal substitution retained their transfection efficiency compared to untargeted 0% substitution LNPs, and interestingly all 3 formulations demonstrated slightly improved transfection over untargeted LNPs without human serum incubation. In neuronal cells, AP2 and mApoE LNPs at 10% substitution maintained a ~6-fold improvement compared to 0% substitution LNPs with and without human serum incubation. Overall, it appears that pLNPs made with 10% lipid-PEG-mal substitution are the most resistant to human serum protein interference.

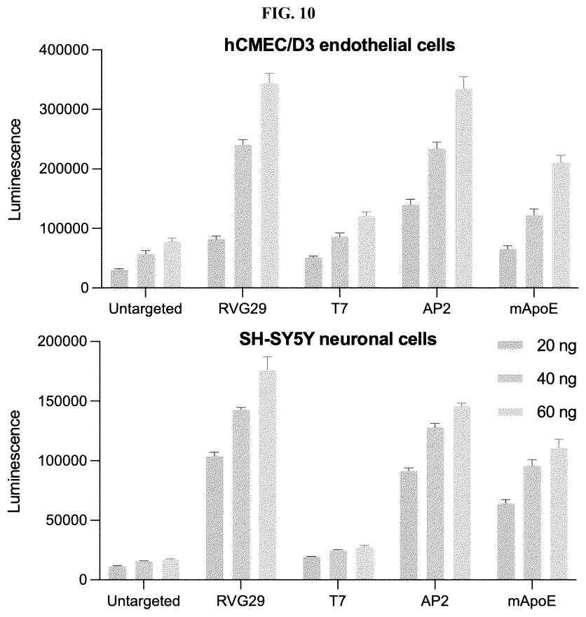

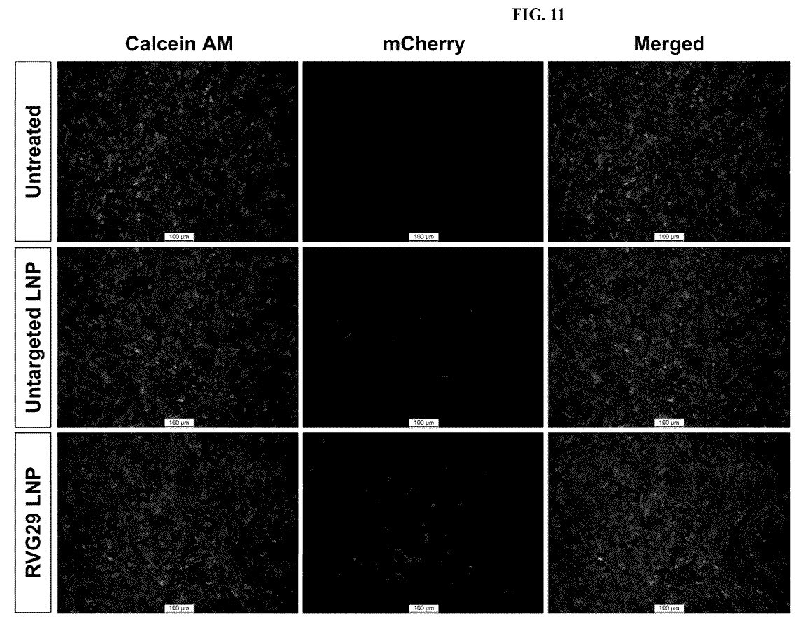

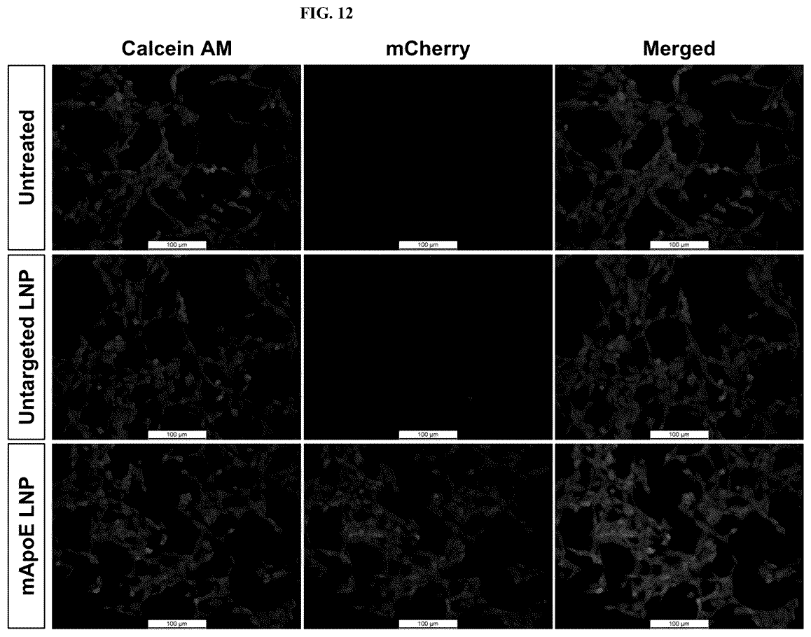

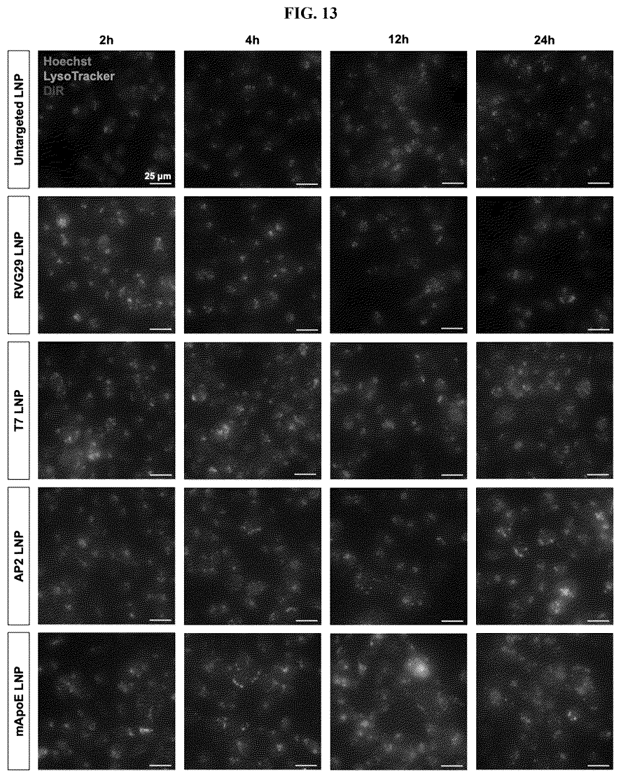

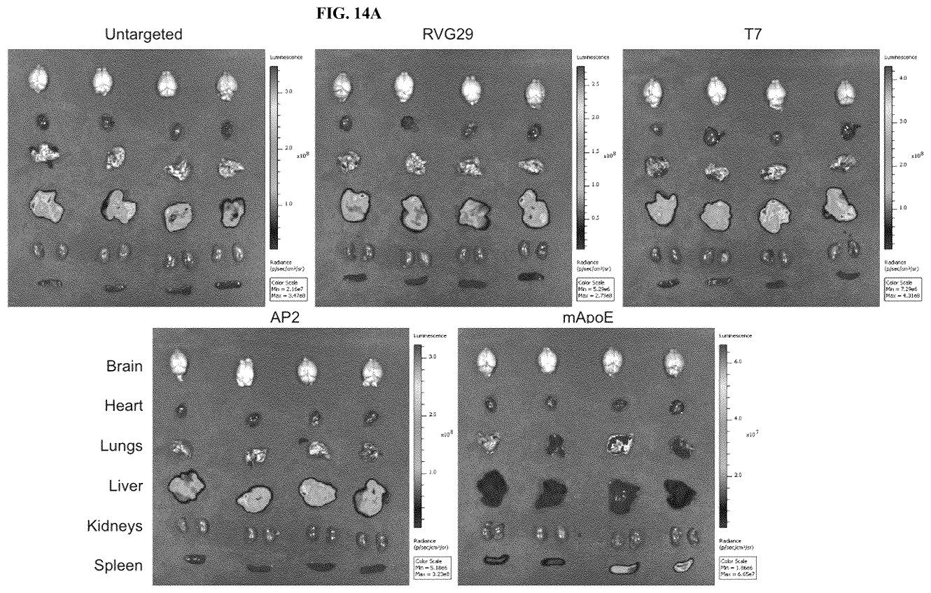

Altogether, these data suggest that LNP targeting benefits conferred by peptide functionalization are sensitive to serum protein interference. Accordingly, serum-containing media were used for the remainder of the in vitro assays to more accurately account for the adsorption of serum proteins. Serum-containing media was also used to confirm that pLNP transfection is dose-dependent (FIG. 10), and that top-performing pLNPs for each cell line can also deliver mCherry mRNA, as visualized with fluorescence microscopy (FIGS. 11-12). Future work should entail detailed characterization of pLNP protein coronas, both before and after BBB transcytosis, as it has been demonstrated that the corona compositions of other nanoparticles change during BBB transcytosis.

### Example 3: Peptide LNPs Enhance BBB Transcytosis and Subsequent Neuronal Transfection In Vitro

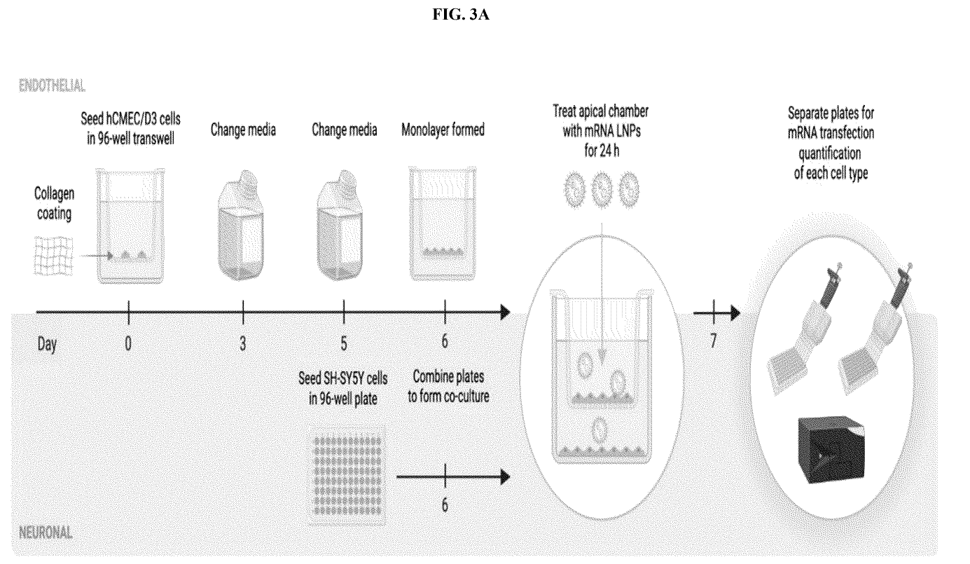

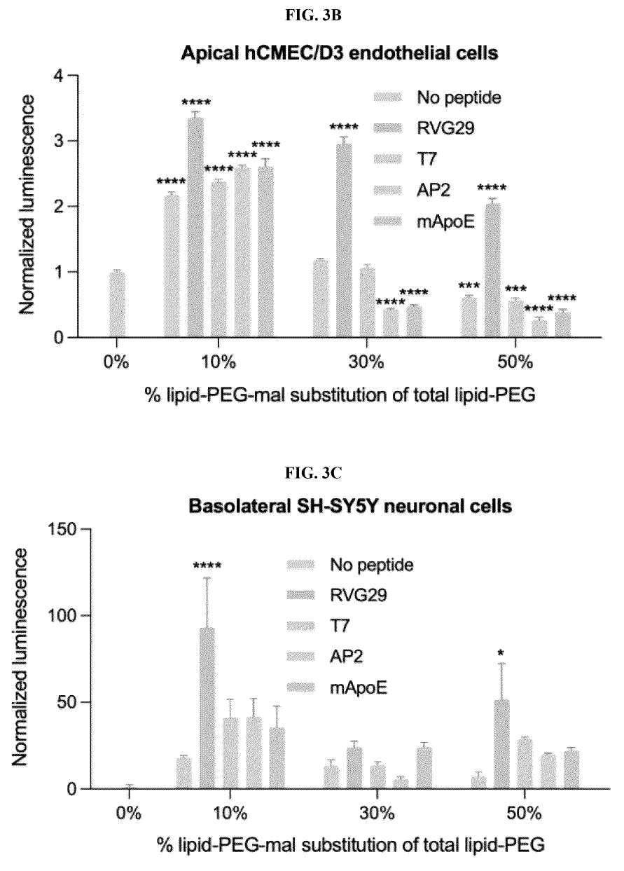

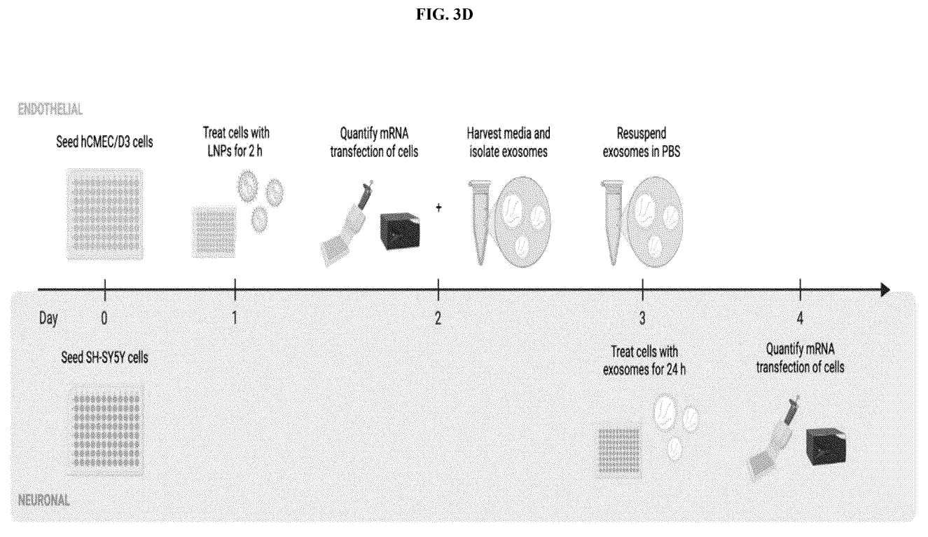

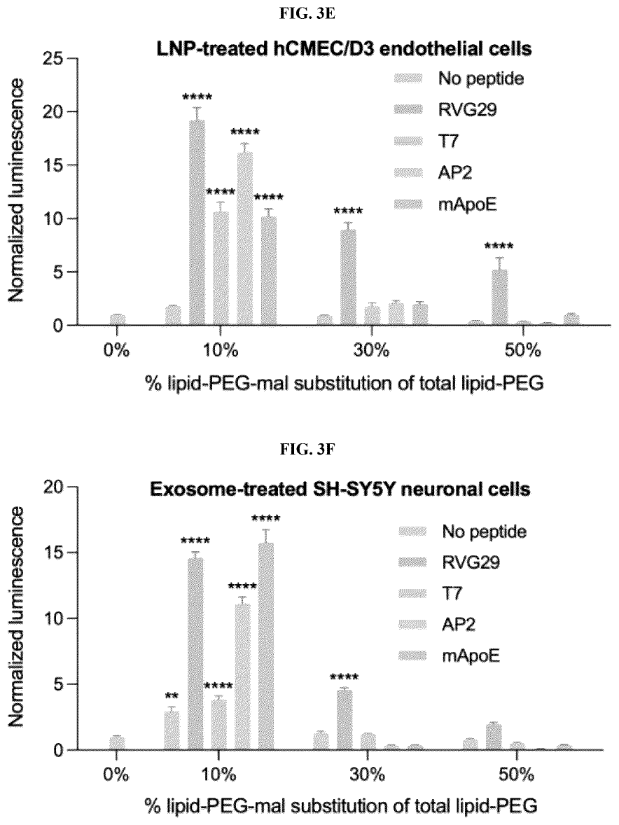

Next, efforts were made to characterize the effect of peptide functionalization on BBB transcytosis. A previously established 96-well transwell model, comprising a brain endothelial monolayer on the transwell insert and neuronal cells in the basolateral compartment, was used and transfection of both cell types was assessed (FIG. 3A). In the apical endothelial cells, pLNPs with 10% lipid-PEG-mal substitution demonstrated the highest transfection, each facilitating a significant increase in luciferase expression compared to 0% substitution LNPs (FIG. 3B). In the 30% and 50% substitution groups, RVG29 LNPs also demonstrated significant increases in transfection compared to 0% substitution LNPs. In basolateral neuronal cells, similar trends were observed, with targeted 10% substitution LNPs mediating the greatest improvement in transfection compared to 0% substitution LNPs, and RVG29 LNPs bearing significant improvements at 10% and 50% substitutions (FIG. 3C). Overall, this data shows that while some pLNPs transfect endothelial cells after endocytosis, others are able to transcytose and transfect neuronal cells. The benefit of peptide targeting is most clear in this transwell model, where 10% substitution RVG29 LNPs mediate an almost 100-fold improvement in neuronal transfection post-BBB crossing.

Next, the mechanisms of LNP endocytosis and transcytosis were further investigated. Fluorescence microscopy was first used to track the intracellular distribution of pLNPs in brain endothelial cells over the course of 24 h. Cells were treated with luciferase mRNA LNPs dyed with DiR for 2 h, and then, after a total of 2, 4, 12, or 24 h of incubation, stained with LysoTracker to visualize acidic compartments like late endosomes and lysosomes (FIG. 13). Yellow signal indicates co-localization of red DiR and green LysoTracker. Cells treated with untargeted LNPs demonstrated low DiR and yellow signal at 2 h, indicating limited LNP uptake. Cells treated with RVG29 LNPs had more yellow signal at 2 h, indicating higher LNP uptake and endosome acidification, as well as diffuse DiR signal at 24 h, indicating endosomal escape. T7 and mApoE LNPs demonstrated the most cellular uptake with bright DiR signal at 12 and 24 h, however the LysoTracker signal was lower than that of RVG29 at 2 h, potentially indicating that the high LNP accumulation inhibited the endosomal acidification necessary for mRNA release and expression. Overall, these results may explain why the 10% substitution RVG29 LNPs facilitated the highest basolateral neuronal transfection in the transwell study. Without wishing to be bound by any theory, these LNPs may enable higher cellular uptake combined with efficient endosome acidification, which allows increased cargo release for both transfection and basolateral secretion.

To further investigate this hypothesis, experiments were conducted to study how peptide functionalization affects the endothelial secretion of LNPs and mRNA cargo. Recent literature has suggested that after LNP endocytosis, mRNA cargo is repackaged and secreted in extracellular vesicles, which then mediate gene transfer to other cells. It was hypothesized that a similar mechanism may occur when LNPs cross the BBB and sought to evaluate this in a proof-of-concept experiment where exosomes were isolated from pLNP-treated endothelial cells (FIG. 3D). In this model, the observed endothelial transfection trends were similar to those from the transwell study, with 10% lipid-PEG-mal substitution pLNPs yielding the highest improvement compared to 0% substitution LNPs, and RVG29 LNPs performing the best across all substitutions (FIG. 3E). The higher magnitudes of improvement compared to earlier experiments is likely a result of the shorter treatment period (2 versus 24 h), as receptor-mediated endocytosis may mediate more rapid cellular uptake. For instance, TfR-targeted immunoliposomes had greater improvements in brain capillary accumulation over untargeted immunoliposomes at 1 h compared to 24 h.

Neuronal cells treated with the isolated exosomes also demonstrated luminescence, indicating that the isolated exosomes encapsulated luciferase mRNA (FIG. 3F). Exosomes derived from 10% substitution pLNP groups significantly improved transfection compared to those from the 0% substitution group. Specifically, exosomes derived from cells treated with RVG29 and mApoE LNPs yielded a ~15-fold improvement.

### Example 4: Peptide LNPs Improve In Vivo Brain Transfection after Systemic Administration

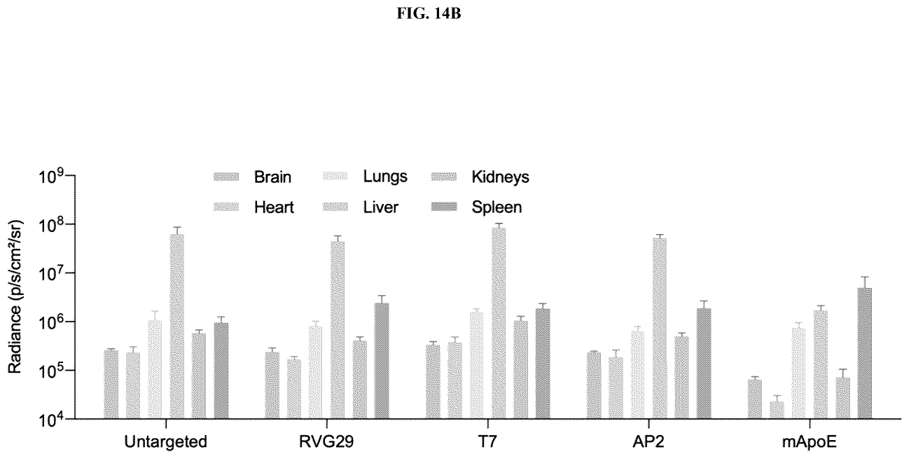

Next, it was evaluated whether peptide targeting could improve in vivo LNP delivery to the brain after systemic administration. Based on the in vitro screening data, 10% lipid-PEG-mal substitution was selected for all pLNPs. LNPs were formulated with luciferase mRNA and intravenously administered to healthy adult C57BL/6 mice at a dose of 0.3 mg/kg. After 6 h, luciferin was intraperitoneally administered, and mice were euthanized for organ collection and ex vivo imaging (FIGS. 14A-14B).

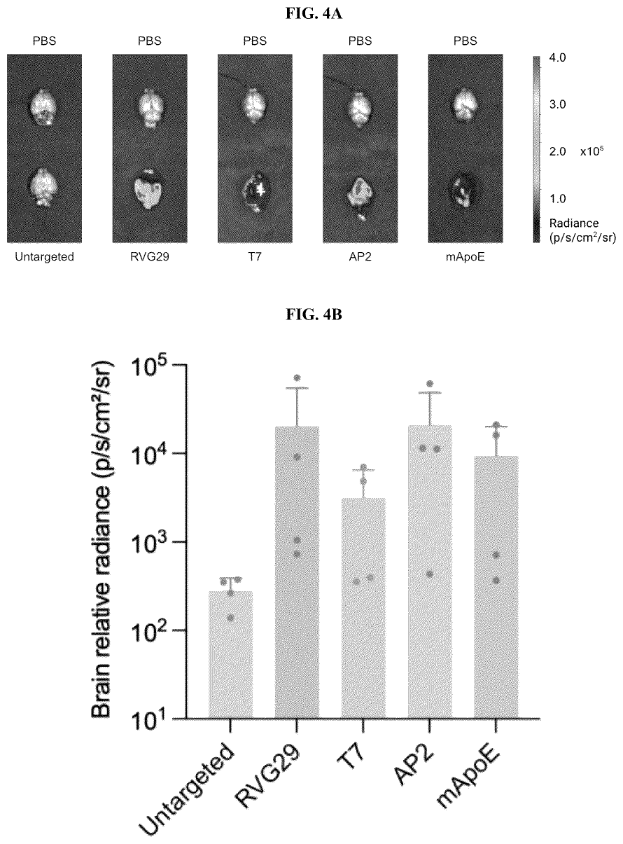

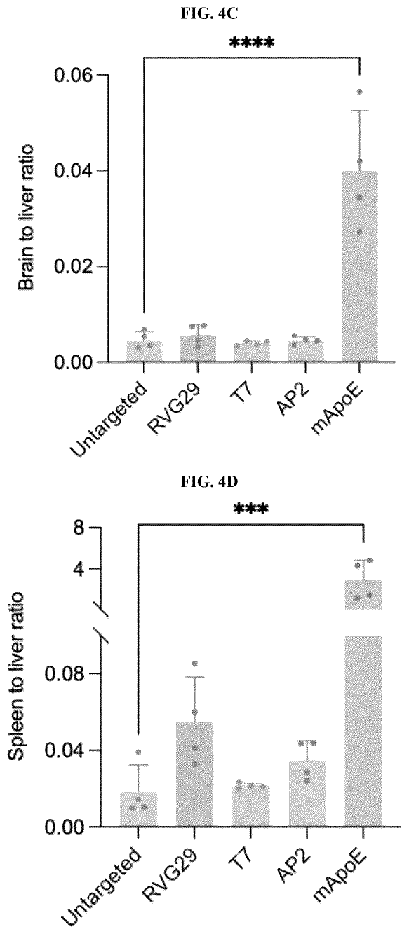

All pLNPs facilitated improvements in brain transfection compared to untargeted LNPs, with RVG29 and AP2 enabling a ~70-fold improvement (FIGS. 4A-4B). The brain-to-liver ratio for mice treated with mApoE LNPs was ~8-fold greater than the those treated with untargeted LNPs (FIG. 4C). This LNP also outperformed other LNPs in extrahepatic delivery, with increased transfection of the spleen and lungs as well (FIGS. 4D-4E). While for all other LNPs at least 90% of the total organ luminescence originated from the liver, for mApoE LNPs, only 25% of signal originated from the liver with the largest contribution being from the spleen (FIG. 4E). Indeed, the spleen-to-liver ratio for mice treated with mApoE LNPs was ~160-fold greater than those treated with untargeted LNPs (FIG. 4D). This is an interesting observation considering that serum ApoE has been shown to drive LNP delivery to hepatocytes via LDLR, as observed with untargeted LNPs (FIG. 4E). This suggests that though the mApoE sequence is derived from ApoE, the two lead to different LNP organ tropisms.

### Example 5: LNP Peptide Targeting Enhances Transfection of Neurons In Vivo

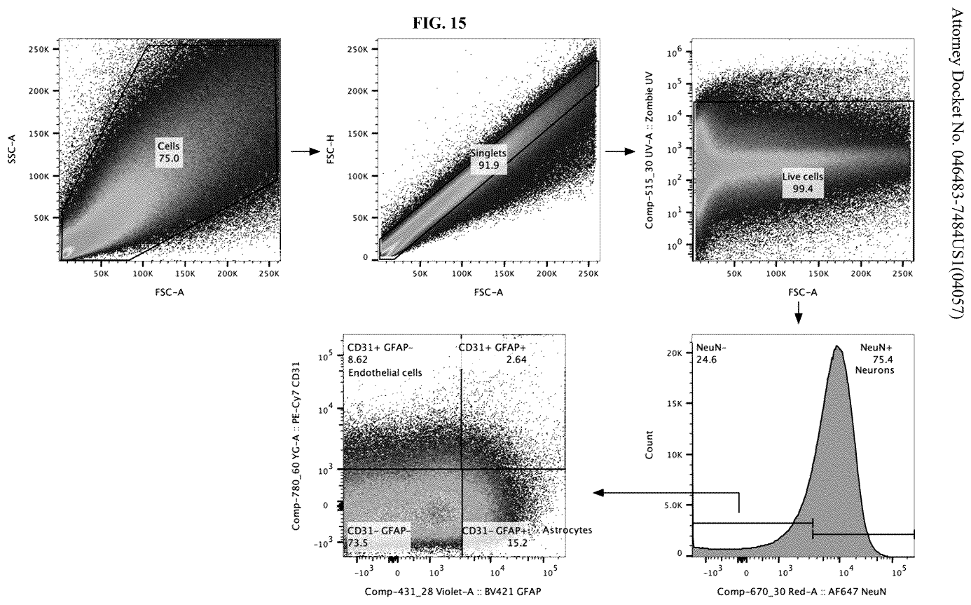

Finally, cell type-specific transfection and uptake in the brain with exemplary pLNP formulations of the disclosure was investigated. LNPs were formulated with mCherry mRNA and 1 mol % DiR and intravenously administered to healthy adult C57BL/6 mice at a dose of 0.3 mg/kg. After 12 h, mice were euthanized, and the brains were processed for flow cytometric analysis. A representative gating strategy is shown in FIG. 15.

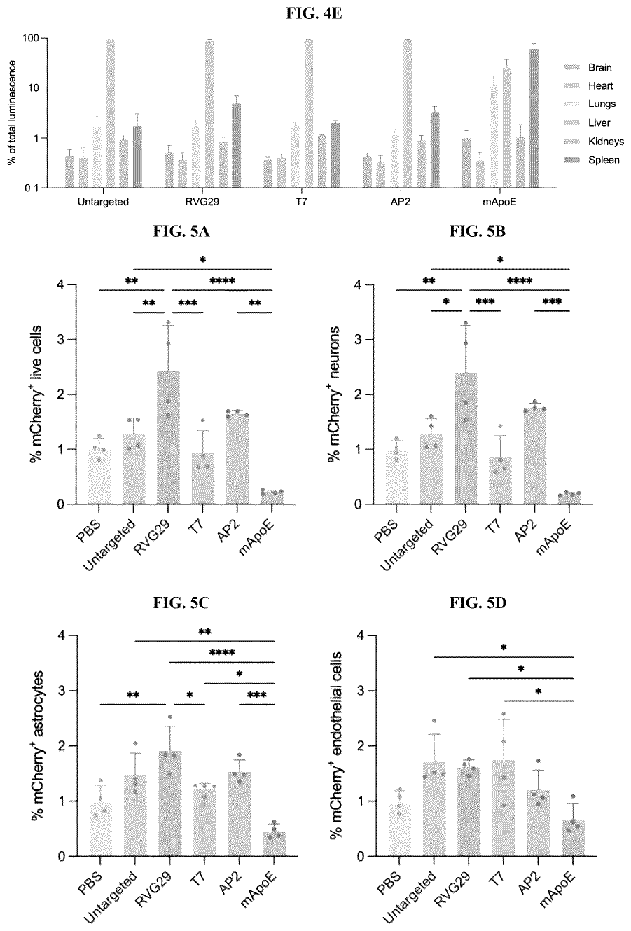

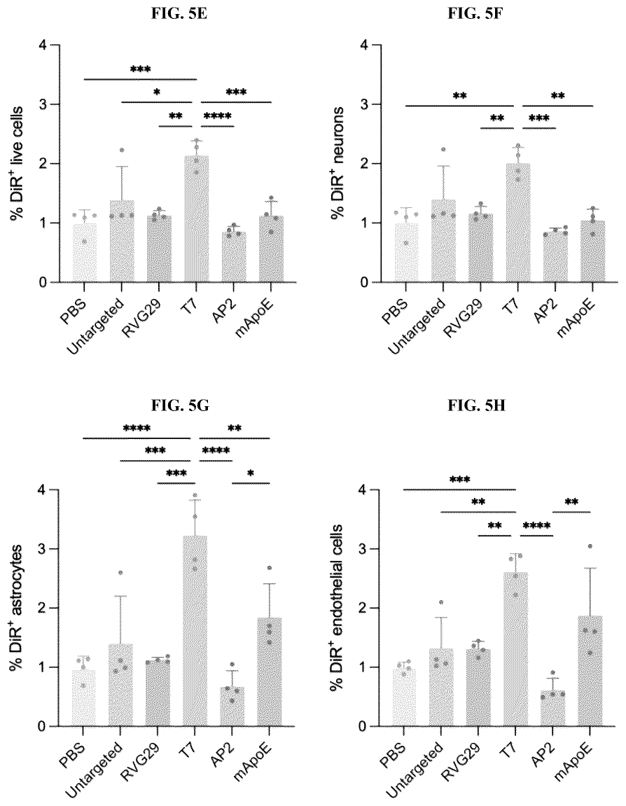

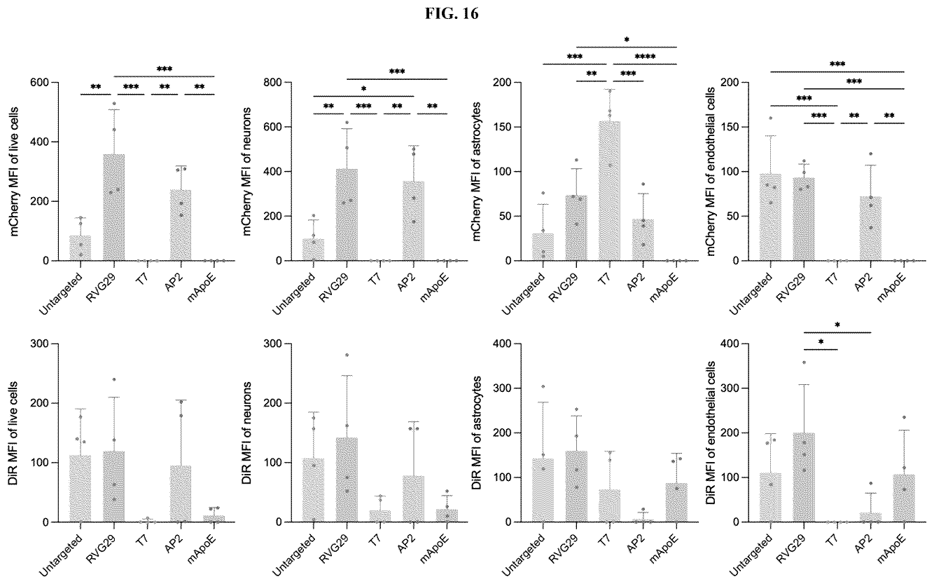

When evaluating all live cells, RVG29 LNPs mediated a significant increase in the percentage of mCherry⁺ cells compared to untargeted LNPs (FIG. 5A). NeuN⁺ neurons from mice treated with RVG29 LNPs were ~2.4% mCherry⁺, whereas neurons from mice treated with untargeted LNPs were only ~1.2% mCherry⁺, which was very close to the background autofluorescence from the PBS-treated group (FIG. 5B). GFAP+/CD31−/NeuN− astrocytes from mice treated with RVG29 LNPs were ~1.9% mCherry⁺, whereas astrocytes from mice treated with untargeted LNPs were ~1.4% mCherry⁺, though this difference was not statistically significant (FIG. 5C). In CD31⁺/GFAP⁻/NeuN⁻ endothelial cells, untargeted, RVG29, and T7 LNPs facilitated similar levels of mCherry transfection (FIG. 5D). Notably, though T7 LNP treatment led to the highest percentage of DiR⁺ live cells, neurons, astrocytes, and endothelial cells compared to other groups (FIGS. 5E-5H), this did not directly correlate to improved transfection. This corroborates what was observed in vitro, where T7 and mApoE LNPs exhibited higher endothelial uptake compared to RVG29 LNPs (FIG. 13) but not higher transfection (FIG. 2B, FIG. 3B, and FIG. 3E). These findings underscore the importance of screening for both transfection and uptake. MFIs for flow cytometry data are shown in FIG. 16.

Together, this data shows that RVG29 LNPs facilitate significant improvements in transfecting neurons but not endothelial cells, suggesting that these targeted formulations are able to cross the BBB and avoid entrapment in endothelial cells. Notably, the identification of RVG29 LNPs as the top performer for neuronal transfection in vivo aligns with the results of the in vitro co-culture transwell study (FIG. 3C), highlighting the utility of this in vitro screening platform for predicting in vivo results.

Sequence Listing
RVG29
(SEQ ID NO: 1)
YTIWMPENPRPGTPCDIFTNSRGKRASNGC
T7
(SEQ ID NO: 2)
CHAIYPRH
Angiopep-2 (AG2)
(SEQ ID NO: 3)
TFFYGGSRGKRNNFKTEEYC
mApoE
(SEQ ID NO: 4)
RLLRKRLKRLGWC

### Enumerated Embodiments

The following exemplary embodiments are provided, the numbering of which is not to be construed as designating levels of importance:
Embodiment 1: A lipid nanoparticle (LNP) composition comprising:
(a) at least one ionizable lipid;
(b) at least one neutral lipid;
(c) cholesterol lipid and/or a modified derivative thereof,
(d) at least one polymer conjugated lipid and/or a modified derivative thereof, and
(e) a cell targeting polypeptide which specifically binds to a receptor overexpressed on the surface of a brain endothelial cell or neuron, optionally wherein the cell targeting domain is covalently conjugated to at least one component of the LNP.
Embodiment 2: The LNP of Embodiment 1, wherein the receptor is at least one of a nicotinic acetylcholine receptor, a transferrin receptor, a low density lipoprotein receptor-related protein 1, and/or a low density lipoprotein receptor (LDLR).
Embodiment 3: The LNP of Embodiment 1 or 2, wherein at least one of the following applies:
(a) the cell targeting polypeptide shares at least 85% sequence homology with RVG29 (SEQ ID NO:1), optionally wherein the cell targeting polypeptide specifically binds to a nicotinic acetylcholine receptor;
(b) the cell targeting polypeptide shares at least 85% sequence homology with T7 (SEQ ID NO:2), optionally wherein the cell targeting polypeptide specifically binds to a transferrin receptor;
(c) the cell targeting polypeptide shares at least 85% sequence homology with angiopep-2 (AP2) (SEQ ID NO:3), optionally wherein the cell targeting polypeptide specifically binds to low density lipoprotein receptor-related protein 1; and
(d) the cell targeting polypeptide shares at least 85% sequence homology with mApoE (SEQ ID NO:4), optionally wherein the cell targeting polypeptide specifically binds to a low density lipoprotein receptor (LDLR).
Embodiment 4: The LNP of any one of Embodiments 1-3, wherein the LNP comprises an ionizable lipid of Formula (I), or a salt, solvate, stereoisomer, or isotopologue thereof:

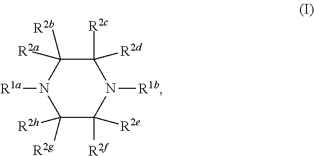

wherein:
R^{1a} and R^{1b} are each independently

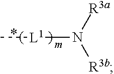

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L¹ is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and each occurrence of m is independently an integer selected from the group consisting of 1, 2, 3, and 4.
Embodiment 5: The LNP of any one of Embodiments 1-4, wherein the at least one ionizable lipid comprises or consists essentially of SM-102:

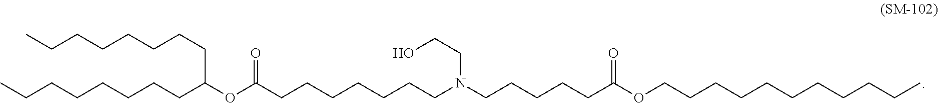 [SMILES: CCCCCCCCCCCOC(=O)CCCCCN(CCO)CCCCCCCC(=O)OC(CCCCCCCC)CCCCCCCC]

Embodiment 6: The LNP of any one of Embodiments 1-5, wherein the at least one ionizable lipid comprises about 10 mol % to about 90 mol % of the LNP, optionally wherein the at least one ionizable lipid comprises about 50 mol % of the LNP.
Embodiment 7: The LNP of any one of Embodiments 1-6, wherein the at least one neutral lipid comprises or consists essentially of distearoylphosphatidylcholine (DSPC).
Embodiment 8: The LNP of any one of Embodiments 1-7, wherein the at least one neutral lipid comprises about 1 mol % to about 45 mol % of the LNP, optionally wherein the at least one neutral lipid comprises about 10 mol % of the LNP.
Embodiment 9: The LNP of any one of Embodiments 1-8, wherein the at least one cholesterol lipid and/or modified derivative thereof comprises or consists essentially of cholesterol.
Embodiment 10: The LNP of any one of Embodiments 1-9, wherein the at least one cholesterol lipid and/or modified derivative thereof comprises about 5 mol % to about 60 mol % of the LNP, optionally wherein at least one cholesterol lipid and/or modified derivative thereof comprises about 38.5 mol % of the LNP.
Embodiment 11: The LNP of any one of Embodiments 1-10, wherein the at least one polymer conjugated lipid and/or modified derivative thereof comprises about 0.5 mol % to about 12.5 mol % of the LNP, optionally wherein the at least one polymer conjugated lipid and/or modified derivative thereof comprises about 1.5 mol % of the LNP.
Embodiment 12: The LNP of any one of Embodiments 1-11, wherein the at least one polymer conjugated lipid and/or modified derivative thereof comprises 1,2-distearoyl-sn-glycero-3-phosphoethanolamine polyethylene glycol 2000 (DSPE-PEG-2000).
Embodiment 13: The LNP of any one of Embodiments 1-12, wherein the cell targeting polypeptide which specifically binds to a receptor overexpressed on the surface of a brain endothelial cell or neuron is covalently conjugated is the modified derivative of the polymer conjugated lipid.
Embodiment 14: The LNP of Embodiment 13, wherein the covalent conjugation comprises a covalent bond forming reaction selected from the group consisting of a [1,4]-conjugate addition (i.e., Michael addition), [4+2]cycloaddition, [3+2]dipolar cycloaddition, nucleophilic addition, transition metal-catalyzed cross-coupling reaction, carbonyl condensation reaction, and reductive amination.
Embodiment 15: The LNP of Embodiment 13 or 14, wherein the covalent conjugation reaction comprises a [1,4]-conjugate addition reaction (i.e., Michael addition).
Embodiment 16: The LNP of Embodiment 14 or 15, wherein the [1,4]-conjugate addition occurs between a maleimide moiety of the modified derivative of the polymer conjugated and a cysteine thiol on the polypeptide.
Embodiment 17: The LNP of any one of Embodiments 1-16, wherein the modified derivative of the polymer conjugated lipid is a compound of Formula (II), or a salt, solvate, stereoisomer, or isotopologue thereof:

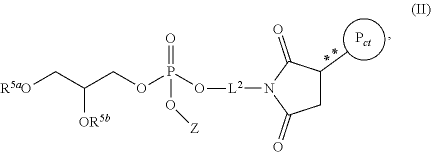

wherein:
R^{5a} and R^{5b} are each independently selected form the group consisting of —C(═O)(optionally substituted C₁-C₂₈ alkyl), —C(═O)(optionally substituted C₂-C₂₈ alkenyl), —C(═O)(optionally substituted C₂-C₂₈ alkynyl), optionally substituted C₁-C₂₈ alkyl, optionally substituted C₂-C₂₈ alkenyl, and optionally substituted C₂-C₂₈ alkynyl;
Z is a monovalent cation;
L² comprises n units of

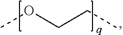

o units of

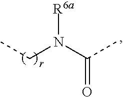

and p units of

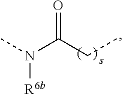

wherein each [symbol] in L² is a C—O or C—N bond;
P_{ct} is a polypeptide which shares at least 8500 sequence homology with a polypeptide selected from the group consisting of RVG29 (SEQ ID NO:1), T7 (SEQ ID NO:2), AP2 (SEQ ID NO:3), and mApoE (SEQ ID NO:4),
   wherein [symbol] is C—S bond;
R^{6a} and R^{6b} are each independently selected from the group consisting of H and C₁-C₆ alkyl;
n, o, and p are each independently 1, 2, 3, 4, or 5;
q is an integer ranging from 1 to 100; and
r and s are each independently an integer ranging from 1 to 10.
Embodiment 18: The LNP of Embodiment 17, wherein Z is NH₄⁺.
Embodiment 19: The LNP of Embodiment 17 or 18, wherein L² is

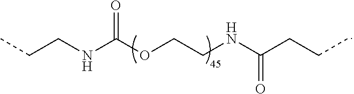

Embodiment 20: The LNP of any one of Embodiments 17-19, wherein the compound of formula (II) is:

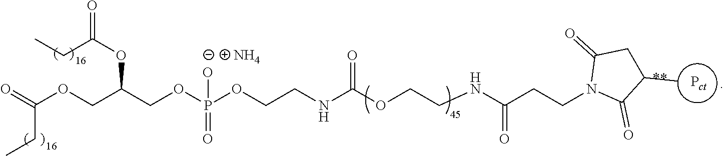

Embodiment 21: The LNP of any one of Embodiments 17-20, wherein (d) comprises the polymer conjugated lipid and the compound of formula (II), wherein the polymer conjugated lipid and the compound of formula (II) have a molar ratio of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10, optionally wherein the molar ratio of polymer conjugated lipid and modified derivative of the conjugated lipid further conjugated to a maleimide moiety is about 2:1.
Embodiment 22: The LNP of any one of Embodiments 1-21, wherein the LNP further comprises at least one cargo selected from the group consisting of a nucleic acid molecule and a therapeutic agent.
Embodiment 23: The LNP of Embodiment 22, wherein the therapeutic agent is at least one selected from the group consisting of a small molecule, a protein, and an antibody.
Embodiment 24: The LNP of Embodiment 23, wherein the LNP comprises a nucleic acid molecule.
Embodiment 25: The LNP of Embodiment 24, wherein the nucleic acid molecule is a DNA molecule or an RNA molecule.
Embodiment 26: The LNP of Embodiment 24 or 25, wherein the nucleic acid molecule is selected from the group consisting of cDNA, mRNA, miRNA, siRNA, modified RNA, antagomir, antisense molecule, and a targeted nucleic acid, or any combination thereof.
Embodiment 27: The LNP of any one of Embodiments 24-26, wherein the nucleic acid molecule encodes a chimeric antigen receptor (CAR).
Embodiment 28: The LNP of Embodiment 27, wherein the CAR is specific for binding to a surface antigen of a pathogenic cell.
Embodiment 29: The LNP of any one of Embodiments 24-28, wherein the nucleic acid molecule encodes at least one selected from the group consisting of mRNA and sgRNA, optionally wherein the ionizable lipid and mRNA have a weight ratio of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or about 1:1.
Embodiment 30: The LNP of Embodiment 29, wherein the mRNA encodes a therapeutic protein, optionally wherein the therapeutic protein is a CRISPR-associated protein, and optionally wherein the CRISPR-associated protein is CRISPR-associated protein 9 (Cas9).
Embodiment 31: The LNP of Embodiment 22, wherein the therapeutic agent is a CRISPR-associated protein, optionally wherein the CRISPR-associated protein is CRISPR-associated protein 9 (Cas9).
Embodiment 32: A pharmaceutical composition comprising the lipid nanoparticle (LNP) of any one of Embodiments 1-31 and at least one pharmaceutically acceptable carrier.
Embodiment 33: A method of treating, preventing, and/or ameliorating a disease or disorder in a subject, the method comprising administering to the subject the LNP of any one of Embodiments 1-31 or the pharmaceutical composition of Embodiment 32.
Embodiment 34: The method of Embodiment 33, wherein the disease or disorder is associated with the brain.
Embodiment 35: The method of Embodiment 33 or 34, wherein the disease or disorder is a neurological disease or disorder.
Embodiment 36: A method of delivering at least one cargo molecule to a brain endothelial cell or neuronal cell of a subject, the method comprising administering to the subject the LNP of any one of Embodiments 1-31 or the pharmaceutical composition of Embodiment 32.
Embodiment 37: The method of any one of Embodiments 33-36, wherein the subject is a mammal.
Embodiment 38: The method of Embodiment 37, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A lipid nanoparticle (LNP) composition comprising:
(a) at least one ionizable lipid;
(b) at least one neutral lipid;
(c) cholesterol lipid or a modified derivative thereof,
(d) at least one polymer conjugated lipid or a modified derivative thereof, and
(e) a cell targeting polypeptide which specifically binds to a receptor overexpressed on the surface of a brain endothelial cell or neuron,
optionally wherein the cell targeting domain is covalently conjugated to at least one component of the LNP.

2. The LNP of claim 1, wherein the receptor is at least one of a nicotinic acetylcholine receptor, a transferrin receptor, a low density lipoprotein receptor-related protein 1, or a low density lipoprotein receptor (LDLR).

3. The LNP of claim 1, wherein at least one of the following applies:
(a) the cell targeting polypeptide shares at least 85% sequence homology with RVG29 (SEQ ID NO:1),
optionally wherein the cell targeting polypeptide specifically binds to a nicotinic acetylcholine receptor;
(b) the cell targeting polypeptide shares at least 85% sequence homology with T7 (SEQ ID NO:2),
optionally wherein the cell targeting polypeptide specifically binds to a transferrin receptor;
(c) the cell targeting polypeptide shares at least 85% sequence homology with angiopep-2 (AP2) (SEQ ID NO:3),
optionally wherein the cell targeting polypeptide specifically binds to low density lipoprotein receptor-related protein 1; and
(d) the cell targeting polypeptide shares at least 85% sequence homology with mApoE (SEQ ID NO:4),
optionally wherein the cell targeting polypeptide specifically binds to a low density lipoprotein receptor (LDLR).

4. The LNP of claim 1, wherein the at least one ionizable lipid comprises or consists essentially of an ionizable lipid of Formula (I), or a salt, solvate, stereoisomer, or isotopologue thereof: 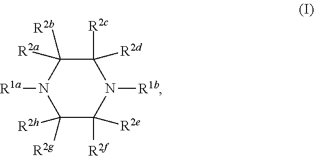
wherein:
R^{1a} and R^{1b} are each independently 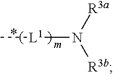
R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L¹ is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently 1, 2, 3, or 4.

5. The LNP of claim 1, wherein the at least one ionizable lipid comprises or consists essentially of SM-102:

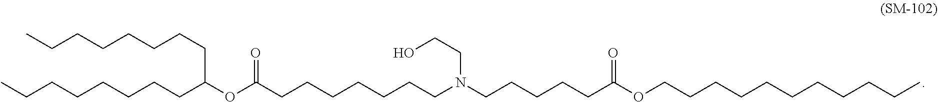 [SMILES: CCCCCCCCCCCOC(=O)CCCCCN(CCO)CCCCCCCC(=O)OC(CCCCCCCC)CCCCCCCC]

6. The LNP of claim 1, wherein at least one of the following applies:
(a) the at least one ionizable lipid comprises about 10 mol % to about 90 mol % of the LNP, optionally wherein the at least one ionizable lipid comprises about 50 mol % of the LNP;
(b) the at least one neutral lipid comprises or consists essentially of distearoylphosphatidylcholine (DSPC);
(c) the at least one neutral lipid comprises about 1 mol % to about 45 mol % of the LNP, optionally wherein the at least one neutral lipid comprises about 10 mol % of the LNP;
(d) the at least one cholesterol lipid or modified derivative thereof comprises or consists essentially of cholesterol;
(e) the at least one cholesterol lipid or modified derivative thereof comprises about 5 mol % to about 60 mol % of the LNP,
optionally wherein at least one cholesterol lipid or modified derivative thereof comprises about 38.5 mol % of the LNP;
(f) the at least one polymer conjugated lipid or modified derivative thereof comprises about 0.5 mol % to about 12.5 mol % of the LNP,
optionally wherein the at least one polymer conjugated lipid or modified derivative thereof comprises about 1.5 mol % of the LNP; and
(g) the at least one polymer conjugated lipid or modified derivative thereof comprises 1,2-distearoyl-sn-glycero-3-phosphoethanolamine polyethylene glycol 2000 (DSPE-PEG-2000).

7. The LNP of claim 1, wherein the cell targeting polypeptide which specifically binds to a receptor overexpressed on the surface of a brain endothelial cell or neuron is covalently conjugated is the modified derivative of the polymer conjugated lipid.

8. The LNP of claim 7, wherein the covalent conjugation comprises a covalent bond forming reaction selected from the group consisting of a [1,4]-conjugate addition (i.e., Michael addition), [4+2]cycloaddition, [3+2]dipolar cycloaddition, nucleophilic addition, transition metal-catalyzed cross-coupling reaction, carbonyl condensation reaction, and reductive amination,
optionally wherein the covalent conjugation reaction comprises a [1,4]-conjugate addition reaction (i.e., Michael addition), and
optionally wherein the [1,4]-conjugate addition occurs between a maleimide moiety of the modified derivative of the polymer conjugated and a cysteine thiol on the polypeptide.

9. The LNP of claim 1, wherein the modified derivative of the polymer conjugated lipid is a compound of Formula (II), or a salt, solvate, stereoisomer, or isotopologue thereof: 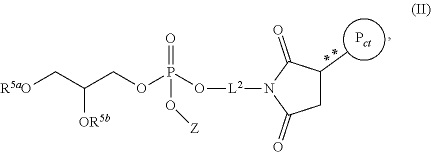
wherein:
R^{5a} and R^{5b} are each independently selected form the group consisting of —C(═O)(optionally substituted C₁-C₂₈ alkyl), —C(═O)(optionally substituted C₂-C₂₈ alkenyl), —C(═O)(optionally substituted C₂-C₂₈ alkynyl), optionally substituted C₁-C₂₈ alkyl, optionally substituted C₂-C₂₈ alkenyl, and optionally substituted C₂-C₂₈ alkynyl;
Z is a monovalent cation;
L² comprises n units of 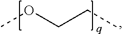
o units of 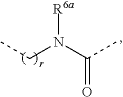
and p units of 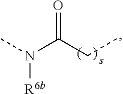
wherein each [symbol] in L² is a C—O or C—N bond;
P_{ct} is a polypeptide which shares at least 85% sequence homology with a polypeptide selected from the group consisting of RVG29 (SEQ ID NO:1), T7 (SEQ ID NO:2), AP2 (SEQ ID NO:3), and mApoE (SEQ ID NO:4),
wherein [symbol] is C—S bond;
R^{6a} and R^{6b} are each independently selected from the group consisting of H and C₁-C₆ alkyl;
n, o, and p are each independently 1, 2, 3, 4, or 5;
q is an integer ranging from 1 to 100; and
r and s are each independently an integer ranging from 1 to 10.

10. The LNP of claim 9, wherein at least one of the following applies:
(a) Z is NH₄⁺; and
(b) L² is

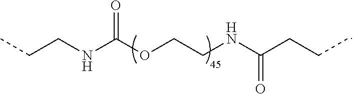

11. The LNP of claim 9, wherein the compound of formula (II) is:

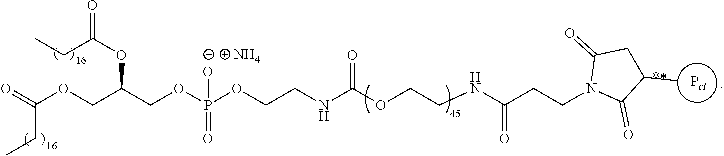

12. The LNP of claim 9, wherein (d) comprises the polymer conjugated lipid and the compound of formula (II), wherein the polymer conjugated lipid and the compound of formula (II) have a molar ratio of about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10,
optionally wherein the molar ratio of polymer conjugated lipid and modified derivative of the conjugated lipid further conjugated to a maleimide moiety is about 2:1.

13. The LNP of claim 1, wherein the LNP further comprises at least one cargo selected from the group consisting of a nucleic acid molecule and a therapeutic agent.

14. The LNP of claim 13, wherein at least one of the following applies:
(a) the therapeutic agent is at least one selected from the group consisting of a small molecule, a protein, and an antibody; and
(b) the LNP comprises a nucleic acid molecule,
optionally wherein the nucleic acid molecule is a DNA molecule or an RNA molecule,
optionally wherein the nucleic acid molecule is selected from the group consisting of cDNA, mRNA, miRNA, siRNA, modified RNA, antagomir, antisense molecule, and a targeted nucleic acid, or any combination thereof.

15. The LNP of claim 13, wherein the nucleic acid molecule encodes a chimeric antigen receptor (CAR),
optionally wherein the CAR is specific for binding to a surface antigen of a pathogenic cell.

16. The LNP of claim 13, wherein the nucleic acid molecule encodes at least one selected from the group consisting of mRNA and sgRNA,
optionally wherein the ionizable lipid and mRNA have a weight ratio of about 20:1, 19:1, 18:1, 17:1, 16:1, 15:1, 14:1, 13:1, 12:1, 11:1, 10:1, 9:1, 8:1,7:1, 6:1, 5:1,4:1,3:1,2:1, or about 1:1.

17. The LNP of claim 16, wherein the mRNA encodes a therapeutic protein,
optionally wherein the therapeutic protein is a CRISPR-associated protein, and
optionally wherein the CRISPR-associated protein is CRISPR-associated protein 9 (Cas9).

18. A pharmaceutical composition comprising the lipid nanoparticle (LNP) of claim 1 and at least one pharmaceutically acceptable carrier.

19. A method of treating, preventing, or ameliorating a disease or disorder in a subject, the method comprising administering to the subject the LNP of claim 1 or a pharmaceutical composition thereof.

20. The method of claim 19, wherein the disease or disorder is associated with the brain.

21. The method of claim 19, wherein the disease or disorder is a neurological disease or disorder.

22. A method of delivering at least one cargo molecule to a brain endothelial cell or neuronal cell of a subject, the method comprising administering to the subject the LNP of claim 1 or a pharmaceutical composition thereof.


SEQUENCE LISTING (4 sequences)

SEQ ID NO: 1 - protein, 30 aa, synthetic construct
YTIWMPENPRPGTPCDIFTNSRGKRASNGC

SEQ ID NO: 2 - protein, 8 aa, synthetic construct
CHAIYPRH

SEQ ID NO: 3 - protein, 20 aa, synthetic construct
TFFYGGSRGKRNNFKTEEYC

SEQ ID NO: 4 - protein, 13 aa, synthetic construct
RLLRKRLKRLGWC